SEQUENCE LISTING (9 sequences)

SEQ ID NO: 1 - DNA, 29684 bp, Synthetic construct
  source 1..29684: mol_type = other DNA
agatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggttttgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaggtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttttaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagcggctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtattgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatataaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattactttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaatctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcacaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtgttgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagtcacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacttacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaatatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacgtacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgttatctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttggaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattatagtgcgtgagccagaagatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttgatgaagtttttaacgccaccagatttgcatctgtttatgcttggaacaggaagagaatcagcaactgtgttgctgattattctgtcctatataatctcgcaccatttttcacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaagcttgattctaaggttagtggtaattataattacctgtatagattgtttaggaagtctaatctcaaaccttttgagagagatatttcaactgaaatctatcaggccggtaacaaaccttgtaatggtgttgcaggttttaattgttactttcctttacgatcatatagtttccgacccacttatggtgttggtcaccaaccatacagagtagtagtactttcttttgaacttctacatgcaccagcaactgtttgtggacctaaaaagtctactaatttggttaaaaacaaatgtgtcaatttcaacttcaatggtttaaaaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaatatgtcaacaactcatatgagtgtgacatacccattggtgcaggtatatgcgctagttatcagactcagactaagtctcatcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaacgtgctttaactggaatagctgttgaacaagacaaaaacacccaagaagtttttgcacaagtcaaacaaatttacaaaacaccaccaattaaatattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaaaggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccataatgcacaagctttaaacacgcttgttaaacaacttagctccaaatttggtgcaatttcaagtgttttaaatgatatcttttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataactctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagataggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcaggttccaacggtactattaccgttgaagagcttaaaaagctccttgaagaatggaacctagtaataggtttcctattccttacatggatttgtcttctacaatttgcctatgccaacaggaataggtttttgtatataattaagttaattttcctctggctgttatggccagtaactttaacttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgcggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgttattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaacttaaatgtctgataatggaccccaaaatcagcgaaatgcactccgcattacgtttggtggaccctcagattcaactggcagtaaccagaatggtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaaacgaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaa

SEQ ID NO: 2 - DNA, 3813 bp, Coronavirus
  source 1..3813: mol_type = other DNA
  CDS 1..3813
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgttatctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttggaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattatagtgcgtgagccagaagatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttgatgaagtttttaacgccaccagatttgcatctgtttatgcttggaacaggaagagaatcagcaactgtgttgctgattattctgtcctatataatctcgcaccatttttcacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaagcttgattctaaggttagtggtaattataattacctgtatagattgtttaggaagtctaatctcaaaccttttgagagagatatttcaactgaaatctatcaggccggtaacaaaccttgtaatggtgttgcaggttttaattgttactttcctttacgatcatatagtttccgacccacttatggtgttggtcaccaaccatacagagtagtagtactttcttttgaacttctacatgcaccagcaactgtttgtggacctaaaaagtctactaatttggttaaaaacaaatgtgtcaatttcaacttcaatggtttaaaaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaatatgtcaacaactcatatgagtgtgacatacccattggtgcaggtatatgcgctagttatcagactcagactaagtctcatcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaacgtgctttaactggaatagctgttgaacaagacaaaaacacccaagaagtttttgcacaagtcaaacaaatttacaaaacaccaccaattaaatattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaaaggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccataatgcacaagctttaaacacgcttgttaaacaacttagctccaaatttggtgcaatttcaagtgttttaaatgatatcttttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 3 - protein, 1270 aa, Coronavirus
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - DNA, 719 bp, Synthetic construct
  source 1..719: mol_type = other DNA
  CDS 1..708
atggtgagcaaaggagaagaggacaacatggcatcactcccagctacacatgagctgcatatcttcggatccatcaacggagtggacttcgatatggtgggacagggtacagggaacccaaacgacggatacgaggagttgaacctgaagagtaccaagggagacctgcagttctcaccatggatactcgtcccacatataggatacggctttcatcagtacctgccctatccagacggaatgtcacctttccaggcagccatggttgacgggagcggttaccaggtccacaggacaatgcagtttgaggacggagcctcattgaccgtgaactacagatatacctacgaaggaagccatatcaagggagaggctcaagtgaagggaactggattcccagcggacggacccgtgatgaccaacagtctgacggctgcagactggtgcagatccaaaaagacctacccaaatgacaagacaatcataagcaccttcaagtggtcatacactacaggaaacgggaagagatacaggagcactgccagaaccacatacactttcgccaagcctatggctgcaaactacctcaagaaccaacccatgtatgtgttcagaaagacagaactgaagcattctaagaccgaactgaacttcaaggagtggcagaaggcctttactgacgtgatgggaatggacgaactctacaagtaattaattaa

SEQ ID NO: 5 - protein, 236 aa, Synthetic construct
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 6 - DNA, 513 bp, Synthetic construct
  source 1..513: mol_type = other DNA
  CDS 1..513
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcg

SEQ ID NO: 7 - protein, 171 aa, Synthetic construct
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 8 - DNA, 1650 bp, Synthetic construct
  source 1..1650: mol_type = other DNA
  CDS 1..1650
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtataccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggtggtaagatcgccgtg

SEQ ID NO: 9 - protein, 550 aa, Synthetic construct
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

(19) United States
(12) Patent Application Publication
(10) Pub. No.: US 2023/0303634 A1
(43) Pub. Date: Sep. 28, 2023

(54) OMICRON SARS-COV-2 ASSAY

(71) Applicant: Board of Regents, The University of Texas System, Austin, TX (US)

(72) Inventors: Hongjie Xia, Galveston, TX (US); Xuping Xie, Galveston, TX (US); Pei-Yong Shi, Galveston, TX (US)

(21) Appl. No.: 18/076,315

(22) Filed: Dec. 6, 2022

Related U.S. Application Data
(60) Provisional application No. 63/307,195, filed on Feb. 7, 2022.

(51) Int. Cl. C07K 14/165; G01N 33/569

(52) U.S. Cl. CPC C07K 14/165; G01N 33/56983; C07K 2319/60

(57) ABSTRACT

Certain embodiments of the invention include recombinant reverse genetic systems for Omicron variant of SARS-CoV-2 virus.

### PRIORITY

This application claims priority to US Provisional Patent 63/307,195 filed February 7, 2022, which is incorporated herein by reference in its entirety.

### STATEMENT REGARDING FEDERALLY FUNDED RESEARCH

This invention was made with government support under HHSN272201600013C, AI134907, AI145617, and UL1TR001439 awarded by the National Institutes of Health (NIH). The government has certain rights in the invention.

### REFERENCE TO SEQUENCE LISTING

A sequence listing required by 37 CFR 1.821-1.825 is being submitted electronically with this application. The sequence listing is incorporated herein by reference.

### BACKGROUND

Severe acute respiratory syndrome coronavirus 2 (SARS-CoV-2) continues to evolve, leading to the emergence of variants of concern (VoC), variants of interest, and variants of monitoring. These variants can increase viral transmission, immune evasion, and/or disease severity (Plante et al., Nature, doi:10.1038/s41586-020-2895-3 2020; Xie et al., Nat Med 27, 620-621, 2021; Liu et al., Nature, doi:10.1101/2021.03.08.434499 2021). The recently emerged Omicron variant (B.1.1.529) was first identified in South Africa on November 2, 2021, and was designated as a new VoC on November 26, along with the four previous VoCs: Alpha, Beta, Gamma, and Delta (URL who.int/news/item/26-11-2021-classification-of-omicron-(b.1.1.529)-sars-cov-2-variant-of-concern, 2021). Since its emergence, Omicron has rapidly spread to over 89 countries, with case doubling in as little as 1.5 to 3 days, leading to global surges of COVID-19 cases (Agency, U. H. S. SARS-CoV-2 variants of concern and variants under investigation in England. Technical brief 31, 2021). Compared to prior variants, the Omicron spike glycoprotein has accumulated more spike mutations, with over 34 mutations, many of which are known to evade antibody neutralization (e.g., K417N, N440K, S477N, E484A and Q493R) or to enhance spike/hACE2 receptor binding (e.g., Q498R, N501Y, and D614G)( Plante et al., Nature, doi:10.1038/s41586-020-2895-3, 2020; Liu et al., Nature, doi:10.1101/2021.03.08.434499, 2021; Chen et al., Nat Med 27, 717-726, 2021; Ku et al., Nature Communications, doi.org/10.1038/s41467-41020-20789-41467, 2021). The high number of spike mutations is associated with decreased potency of antibody therapy and increased breakthrough Omicron infections in vaccinated and previously infected individuals (Agency, U. H. S. SARS-CoV-2 variants of concern and variants under investigation in England. Technical brief 31, 2021). Laboratory studies are urgently needed to examine the susceptibility of Omicron SARS-CoV-2 to vaccine-elicited and infection-elicited neutralization. There is a need for methods and compositions for examination of cross-neutralization of Omicron, for example cross-neutralization by antibodies derived from previous non-Omicron infection.

### SUMMARY

The need for additional assays for characterization of Omicron is addressed by the compositions and assays described herein. Certain embodiments are directed to a recombinant SARS-CoV-2 nucleic acid segment encoding a heterologous Coronavirus S protein and a reporter protein. In certain aspects the heterologous Coronavirus S protein is the S protein encoded by the Omicron Coronavirus variant. In certain aspects the reporter protein replaces the ORF7a encoding segment of Coronavirus. The reporter protein can be, for example but not limited to mNeonGreen (e.g., SEQ ID NO:4 and SEQ ID NO:5) or nanoluciferase reporter (e.g., SEQ ID NO:6 and SEQ ID NO:7)). The recombinant SARS-CoV-2 can be encoded by SEQ ID NO:1. In certain aspects the nucleic acid segment encoding the heterologous S protein has a nucleic acid sequence that is at least 95, 96, 97, 98, 99, to 100% identical to the nucleic acid sequence

atgtttgtttttcttgttttattgccactagtctctagtcagtgtgttaa
tcttacaaccagaactcaattaccccctgcatacactaattctttcacac
gtggtgtttattaccctgacaaagttttcagatcctcagttttacattca
actcaggacttgttcttacctttcttttccaatgttacttggttccatgt
tatctctgggaccaatggtactaagaggtttgataaccctgtcctaccat
ttaatgatggtgtttattttgcttccattgagaagtctaacataataaga
ggctggatttttggtactactttagattcgaagacccagtccctacttat
tgttaataacgctactaatgttgttattaaagtctgtgaatttcaatttt
gtaatgatccatttttggaccacaaaaacaacaaaagttggatggaaagt
gagttcagagtttattctagtgcgaataattgcacttttgaatatgtctc
tcagccttttcttatggaccttgaaggaaaacagggtaatttcaaaaatc
ttagggaatttgtgtttaagaatattgatggttattttaaaatatattct
aagcacacgcctattatagtgcgtgagccagaagatctccctcagggttt
ttcggctttagaaccattggtagatttgccaataggtattaacatcacta
ggtttcaaactttacttgctttacatagaagttatttgactcctggtgat
tcttcttcaggttggacagctggtgctgcagcttattatgtgggttatct
tcaacctaggacttttctattaaaatataatgaaaatggaaccattacag
atgctgtagactgtgcacttgaccctctctcagaaacaaagtgtacgttg
aaatccttcactgtagaaaaaggaatctatcaaacttctaactttagagt
ccaaccaacagaatctattgttagatttcctaatattacaaacttgtgcc
cttttgatgaagtttttaacgccaccagatttgcatctgtttatgcttgg
aacaggaagagaatcagcaactgtgttgctgattattctgtcctatataa
tctcgcaccatttttcacttttaagtgttatggagtgtctcctactaaat
taaatgatctctgctttactaatgtctatgcagattcatttgtaattaga
ggtgatgaagtcagacaaatcgctccagggcaaactggaaatattgctga
ttataattataaattaccagatgattttacaggctgcgttatagcttgga
attctaacaagcttgattctaaggttagtggtaattataattacctgtat
agattgtttaggaagtctaatctcaaaccttttgagagagatatttcaac
tgaaatctatcaggccggtaacaaaccttgtaatggtgttgcaggtttta
attgttactttcctttacgatcatatagtttccgacccacttatggtgtt
ggtcaccaaccatacagagtagtagtactttcttttgaacttctacatgc
accagcaactgtttgtggacctaaaaagtctactaatttggttaaaaaca
aatgtgtcaatttcaacttcaatggtttaaaaggcacaggtgttcttact
gagtctaacaaaaagtttctgcctttccaacaatttggcagagacattgc
tgacactactgatgctgtccgtgatccacagacacttgagattcttgaca
ttacaccatgttcttttggtggtgtcagtgttataacaccaggaacaaat
acttctaaccaggttgctgttctttatcagggtgttaactgcacagaagt
ccctgttgctattcatgcagatcaacttactcctacttggcgtgtttatt
ctacaggttctaatgtttttcaaacacgtgcaggctgtttaataggggct
gaatatgtcaacaactcatatgagtgtgacatacccattggtgcaggtat
atgcgctagttatcagactcagactaagtctcatcggcgggcacgtagtg
tagctagtcaatccatcattgcctacactatgtcacttggtgcagaaaat
tcagttgcttactctaataactctattgccatacccacaaattttactat
tagtgttaccacagaaattctaccagtgtctatgaccaagacatcagtag
attgtacaatgtacatttgtggtgattcaactgaatgcagcaatcttttg
ttgcaatatggcagtttttgtacacaattaaaacgtgctttaactggaat
agctgttgaacaagacaaaaacacccaagaagtttttgcacaagtcaaac
aaatttacaaaacaccaccaattaaatattttggtggttttaatttttca
caaatattaccagatccatcaaaaccaagcaagaggtcatttattgaaga
tctacttttcaacaaagtgacacttgcagatgctggcttcatcaaacaat
atggtgattgccttggtgatattgctgctagagacctcatttgtgcacaa
aagtttaaaggccttactgttttgccacctttgctcacagatgaaatgat
tgctcaatacacttctgcactgttagcgggtacaatcacttctggttgga
cctttggtgcaggtgctgcattacaaataccatttgctatgcaaatggct
tataggtttaatggtattggagttacacagaatgttctctatgagaacca
aaaattgattgccaaccaatttaatagtgctattggcaaaattcaagact
cactttcttccacagcaagtgcacttggaaaacttcaagatgtggtcaac
cataatgcacaagctttaaacacgcttgttaaacaacttagctccaaatt
tggtgcaatttcaagtgttttaaatgatatcttttcacgtcttgacaaag
ttgaggctgaagtgcaaattgataggttgatcacaggcagacttcaaagt
ttgcagacatatgtgactcaacaattaattagagctgcagaaatcagagc
ttctgctaatcttgctgctactaaaatgtcagagtgtgtacttggacaat
caaaaagagttgatttttgtggaaagggctatcatcttatgtccttccct
cagtcagcacctcatggtgtagtcttcttgcatgtgacttatgtccctgc
acaagaaaagaacttcacaactgctcctgccatttgtcatgatggaaaag
cacactttcctcgtgaaggtgtctttgtttcaaatggcacacactggttt
gtaacacaaaggaatttttatgaaccacaaatcattactacagacaacac
atttgtgtctggtaactgtgatgttgtaataggaattgtcaacaacacag
tttatgatcctttgcaacctgaattagactcattcaaggaggagttagat
aaatattttaagaatcatacatcaccagatgttgatttaggtgacatctc
tggcattaatgcttcagttgtaaacattcaaaaagaaattgaccgcctca
atgaggttgccaagaatttaaatgaatctctcatcgatctccaagaactt
ggaaagtatgagcagtatataaaatggccatggtacatttggctaggttt
tatagctggcttgattgccatagtaatggtgacaattatgctttgctgta
tgaccagttgctgtagttgtctcaagggctgttgttcttgtggatcctgc
tgcaaatttgatgaagacgactctgagccagtgctcaaaggagtcaaatt
acattacacataa (SEQ ID NO:2).

The encoded heterologous S protein can have an amino acid sequence that is at least 95, 96, 97, 98, 99, to 100% identical to

MFVFLVLLPLVSSQCVNLTTRTQLPPAYTNSFTRGVYYPDKVFRSSVLHS
TQDLFLPFFSNVTWFHVISGTNGTKRFDNPVLPFNDGVYFASIEKSNIIR
GWIFGTTLDSKTQSLLIVNNATNVVIKVCEFQFCNDPFLDHKNNKSWMES
EFRVYSSANNCTFEYVSQPFLMDLEGKQGNFKNLREFVFKNIDGYFKIYS
KHTPIIVREPEDLPQGFSALEPLVDLPIGINITRFQTLLALHRSYLTPGD
SSSGWTAGAAAYYVGYLQPRTFLLKYNENGTITDAVDCALDPLSETKCTL
KSFTVEKGIYQTSNFRVQPTESIVRFPNITNLCPFDEVFNATRFASVYAW
NRKRISNCVADYSVLYNLAPFFTFKCYGVSPTKLNDLCFTNVYADSFVIR
GDEVRQIAPGQTGNIADYNYKLPDDFTGCVIAWNSNKLDSKVSGNYNYLY
RLFRKSNLKPFERDISTEIYQAGNKPCNGVAGFNCYFPLRSYSFRPTYGV
GHQPYRVVVLSFELLHAPATVCGPKKSTNLVKNKCVNFNFNGLKGTGVLT
ESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGA
EYVNNSYECDIPIGAGICASYQTQTKSHRRARSVASQSIIAYTMSLGAEN
SVAYSNNSIAIPTNFTISVTTEILPVSMTKTSVDCTMYICGDSTECSNLL
LQYGSFCTQLKRALTGIAVEQDKNTQEVFAQVKQIYKTPPIKYFGGFNFS
QILPDPSKPSKRSFIEDLLFNKVTLADAGFIKQYGDCLGDIAARDLICAQ
KFKGLTVLPPLLTDEMIAQYTSALLAGTITSGWTFGAGAALQIPFAMQMA
YRFNGIGVTQNVLYENQKLIANQFNSAIGKIQDSLSSTASALGKLQDVVN
HNAQALNTLVKQLSSKFGAISSVLNDIFSRLDKVEAEVQIDRLITGRLQS
LQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWF
VTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSC
CKFDEDDSEPVLKGVKLHYT SEQ ID NO:3.

In certain aspects, the encoded heterologous S protein has an amino acid sequence of SEQ ID NO:3. In certain aspects the heterologous S protein can include any combination of substitution(s), insertion(s) or deletion(2) including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more amino acid substitutions or variants with respect to SEQ ID NO:3; a 1, 2, 3, 4, 5 deletion of consecutive amino acids, and/or a 1, 2, 3, 4, 5, amino acid insertion. Thus, theheterologous S protein can be an S protein variant. The recombinant SARS-CoV-2 nucleic acid segment can be at least 95, 96, 97, 98, 99, to 100% identical to the nucleic acid sequence of SEQ ID NO:1. In certain aspects the expression cassette is comprised in a plasmid backbone. The SARS-CoV-2 nucleic acid segment can be operatively coupled to a heterologous promoter segment.

Certain embodiments are directed to host cells comprising an expression cassette as described herein or a recombinant SARS-CoV-2 RNA transcribed from an expression cassette described herein.

Other embodiments are directed to recombinant SARS-CoV-2 genomes comprising a nucleic acid sequence encoding a heterologous S protein and a reporter protein replacing an ORF7a encoding segment. The reporter protein can be a fluorescent or luminescent protein, or other detectable polypeptide or transcript. In certain aspects the fluorescent protein is a mNeonGreen protein. In certain aspects the luminescent protein is a nanoluciferase protein.

Certain embodiments are directed to a recombinant cDNA encoding a heterologous S protein and a reporter protein replacing an ORF7a. In certain aspects the recombinant cDNA comprises a nucleotide sequence that is at least 95, 96, 97, 98, 99 to 100% identical to SEQ ID NO:1.

Other embodiments are directed to assays for SARS-CoV-2 replication comprising: (i) contacting a cultured cell expressing or containing a SARS-CoV-2 nucleotide sequence as described herein forming a test cell; (ii) contacting the test cell with a test agent; and (iii) assessing the replication of the SARS-CoV-2 in the presence of the test agent. In certain aspects the cultured cell is a Vero cell. The cultured cell can be assayed in a multi-well plate. In certain aspects the multi-well plate is a 96 well microtiter plate. The cells can be incubated for about 12, 24, 36, or 48 hours before measuring the reporter signal.

The term “coronavirus” refers to a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. Coronavirus RNAs can encode: (1) an RNA-dependent RNA polymerase; (2) N-protein; (3) three envelope glycoproteins; and (4) three non-structural proteins. These coronaviruses infect a variety of mammals and birds. They cause respiratory infections (common), enteric infections (mostly in infants >12 mo.), and possibly neurological syndromes. Coronaviruses are transmitted by aerosols of respiratory secretions. Coronaviruses are exemplified by, but not limited to, human enteric SARS-CoV-2 (GenBank accession number NC_045512.2), coV (ATCC accession # VR-1475), human coV 229E (ATCC accession # VR-740), human coV OC43 (ATCC accession # VR-920), and SARS-coronavirus (Center for Disease Control).

The term “nucleic acid” refers to a polymeric compound comprising nucleosides or nucleoside analogs which have nitrogenous heterocyclic bases or base analogs linked together to form a polynucleotide, including conventional RNA, DNA, mixed RNA-DNA, and polymers that are analogs thereof. A nucleic acid “backbone” may be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid may be ribose, deoxyribose. Nitrogenous bases may be conventional bases (A, G, C, T, U), analogs thereof (e.g., inosine or others; see The Biochemistry of the Nucleic Acids 5-36, Adams et al., ed., 11th ed., 1992)

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

The term “recombinant” refers to an artificial combination of two otherwise separated segments of nucleic acid, e.g., by chemical synthesis or by the manipulation of isolated segments of nucleic acids by genetic engineering techniques.

The term “SARS-CoV-2 replicon” is used to refer to a nucleic acid molecule expressing SARS-CoV-2 genes such that it can direct its own replication (amplification).

The term “SARS-CoV-2 replicon particle” refers to a virion or virion-like structural complex incorporating a SARS-CoV-2 replicon.

The term “SARS-CoV-2 reporter virus” refers to a virus that is capable of directing the expression of a sequence(s) or gene(s) of interest. The reporter construct can include a 5′ sequence capable of initiating transcription of a nucleic acid encoding a reporter molecule or protein such as luciferase, fluorescent protein, Neo, SV2 Neo, hygromycin, phleomycin, histidinol, and DHFR. The reporter virus can be used an indicator of infection of a cell by a SARS-CoV-2 virus.

The term “expression vector” refers to a nucleic acid that is capable of directing the expression of a sequence(s) or gene(s) of interest. The vector construct can include a 5′ sequence capable of initiating transcription of a nucleic acid, e.g., all or part of a SARS-CoV-2 virus. The vector may also include nucleic acid molecule(s) to allow for production of virus, a 5′ promoter that is capable of initiating the synthesis of viral RNA in vitro from cDNA, as well as one or more restriction sites, and a polyadenylation sequence. In addition, the constructs may contain selectable markers such as Neo, SV2 Neo, hygromycin, phleomycin, histidinol, and DHFR. Furthermore, the constructs can include plasmid sequences for replication in host cells and other functionalities known in the art. In certain aspects the vector construct is a DNA construct.

The term “expression cassette” refers to a nucleic acid segment capable of directing the expression of one or more nucleic acids, or one or more nucleic acids that are in turn translated into an expressed protein.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1B. Reduced neutralization of Omicron SARS-CoV-2 by previous non-Omicron viral infection. 50% fluorescent focus reduction neutralization titers (FFRNT₅₀) were measured for two serum panels from patients previously infected with non-Omicron SARS-CoV-2. The first serum panel was collected at 1-month post-infection (n=64) and the second panel collected at 6-months post-infection (n=36). For each serum, FFRNT₅₀ values were determined against mNG USA-WA1/2020 and Omicron-spike SARS-CoV-2. (A) FFRNT₅₀s of 1-month post-infection sera. (1B) FFRNT₅₀s of 6-month post-infection sera. Table 1 and Table 2 summarize the FFRNT₅₀ values and serum information for (A) and (B), respectively. Each symbol of dots (A) and triangles (B) represents one serum specimen. The FFRNT₅₀ value for each serum was determined in duplicate assays and is presented as the geometric mean. The bar heights and the numbers above each set of data indicate geometric mean titers. The whiskers indicate 95% confidence intervals. The dotted line indicates the first serum dilution (1:20) of the FFRNT assay. The FFRNT₅₀ values of sera that did not show any inhibition of viral infection are presented as 10 for plot purposes and statistical analysis. Statistical analysis was performed using the Wilcoxon matched-pairs signed-rank test. The statistical significance of the difference between the geometric mean titers against USA-WA1/2020 and Omicron-spike SARS-CoV-2 is p <0.0001 in both (A) and (B).

FIG. 2. Construction of mNeonGreen (mNG) Omicron-spike SARS-CoV-2. mNG USA-WA1/2020 was used to engineer the complete spike gene from the Omicron variant, resulting in mNG Omicron-spike SARS-CoV-2. Mutations (red circle), deletions (x), and insertions (+) are indicated. Nucleotide and amino acid positions are depicted. L: leader sequence; ORF: open reading frame; RBD: receptor binding domain; S: spike glycoprotein; S1: N-terminal furin cleavage fragment of S; S2: C-terminal furin cleavage fragment of S; E: envelope protein; M: membrane protein; N: nucleoprotein; UTR: untranslated region.

FIG. 3. Fluorescent foci of mNG USA-WA1/2020 and mNG Omicron-spike SARS-CoV-2 on Vero E6 cells. Original and processed images were collected by high-content imaging. The protocol of the fluorescent focus reduction neutralization test (FFRNT) is described in Methods. See FIG. 4 for the experimental scheme of FFRNT.

FIG. 4 Experimental scheme of fluorescent focus reduction neutralization test (FFRNT). The FFRNT protocol is described in Methods.

FIGS. 5A-5B. Reduced neutralization against Omicron SARS-CoV-2 by previous non-Omicron viral infection. 50% fluorescent focus reduction neutralization titers (FFRNT₅₀) were measured for two serum panels from COVID-19 patients previously infected with non-Omicron SARS-CoV-2. The first serum panel was collected at 1-month post-infection (n=64) and the second panel collected at 6-month post-infection (n=36). For each serum, two FFRNT₅₀ values against mNG USA-WA1/2020 and Omicron-spike SARS-CoV-2 are connected by a line. (A) FFRNT₅₀s of 1-month post-infection sera. (B) FFRNT₅₀s of 6-month post-infection sera. Table 1 and Table 2 summarize the FFRNT₅₀ values and serum information for (a) and (b), respectively.

FIGS. 6A-6B. FFRNT₅₀ of 6 pairs of 1- and 6-month post-infection sera from same patients. (A) FFRNT₅₀s of 1-month post-infection sera against mNG USA-WA1/2020 and Omicron-spike SARS-CoV-2. (B) FFRNT₅₀s of 6-month post-infection sera against mNG USA-WA1/2020 and Omicron-spike SARS-CoV-2.

FIGS. 7A-7D. Neutralization of Omicron sublineages before and after 4 doses of mRNA vaccine. (A) Construction of Omicron sublineage-spike mNG SARS-CoV-2. mNG USA-WA1/2020 SARS-CoV-2 was used to engineer Omicron-spike SARS-CoV-2s. The mNG reporter gene was engineered at the open-reading-frame-7 (ORF7) of the USA-WA1/2020 genome.1 Amino acid mutations, deletions (Δ), and insertions (Ins) are indicated for variant spikes in reference to the USA-WA1/2020 spike. L: leader sequence; ORF: open reading frame; NTD: N-terminal domain of S1; RBD: receptor binding domain of S1; S: spike glycoprotein; S1: N-terminal furin cleavage fragment of S; S2: C-terminal furin cleavage fragment of S; E: envelope protein; M: membrane protein; N: nucleoprotein; UTR: un-translated region. Twenty-five pairs of human sera were collected 3-8 months after dose 3 and 1-3 months after dose 4 mRNA vaccine. The FFRNT50s for mNG BA.1-, BA.2-, BA.2.12.1, BA.3-, and BA.4/5-spike SARS-CoV-2s were determined in duplicate assays; the FFRNT50 for USA-WA1/2020 SARS-CoV-2 was determined in two independent ex-periments, each with duplicate assays. (B) FFRNT50 of sera collected before dose 4 vaccine. The bar heights and the numbers above indicate neutralizing GMTs. The whisk-ers indicate 95% CI. The fold of GMT reduction against each Omicron sublineage, com-pared with the GMT against USA-WA1/2020, is shown in italic font. The dotted line indi-cates the limit of detection of FFRNT50. FFRNT50 of <20 was treated as 10 for plot pur-pose and statistical analysis. The p values (Wilcoxon matched-pairs signed-rank test) for group comparison of GMTs are the following. USA-WA1/2020 versus all Omicron sublineage-spike: <0.0001; BA.1-spike versus BA.2-, BA.2.12.1-, BA.3-, BA.4/5-spike: 0.004, 0.0336, <0.0001, < 0.0001, respectively; BA.2-spike versus BA.2.12.1-, BA.3-, BA.4/5-spike: 0.5, 0.065, 0.0083, respectively. BA.2.12.1-spike versus BA.3-, BA.4/5-spike: 0.0098, 0.0002, respectively; BA.3-spike versus BA.4/5-spike: 0.156. (C) FFRNT50 of sera collected after dose 4 vaccine. The p values (Wilcoxon matched-pairs signed-rank test) for group comparison of GMTs are the following. USA-WA1/2020 versus all Omi-cron sublineage-spike: <0.0001; BA.1-spike versus BA.2-, BA.2.12.1-, BA.3-, BA.4/5, BA.2.75-spike: 0.008, 0.033, <0.0001, < 0.0001, 0.37, respectively; BA.2-spike versus BA.2.12.1-, BA.3-, BA.4/5, BA.2.75-spike: 0.12, <0.0001, < 0.0001, 0.56, respectively; BA.2.12.1-spike versus BA.3-, BA.4/5, BA.2.75-spike: 0.0002, <0.0001, 0.94, respective-ly; BA.3-spike versus BA.4/5-, BA.2.75-spike: 0.0009, 0.13, respectively; BA.4/5-spike versus BA.2.75-spike: 0.017. (D) FFRNT50 values with connected lines for each serum pair before and after dose 4 vaccine. The GMT fold increase before and after dose 4 is shown in italic font. The p values of GMT (Wilcoxon matched-pairs signed-rank test) be-fore and after dose 4 vaccines are all <0.0001. The p values(Friedman with Dunn′s multiple comparisons test) for group comparison of the increase in neutralization of sera against different variants are the fllowing. BA.4/5-spike (5.6 folds) verus WA1 (10.8 folds), BA.1-spike (11.2 folds), BA.2-spike (9.8 folds): 0.042, 0.0007, 0.048, respectively.

FIGS. 8A-8B. Neutralization of Omicron sublineages after 2 or 3 doses of mRNA vaccine and BA.1 in-fection. (A) FFRNT50 of 2-dose-vaccine-plus-BA.1-infection sera. Twenty-nine sera were collected from individuals who received 3 doses of vaccine and subsequently contracted BA.1 breakthrough infection. The GMT reduction fold against each Omicron sublineage and USA-WA1/2020 is shown in italic font. The dotted line indicates the limit of detection of FFRNT50. FFRNT50 of <20 was treated as 10 for plot purpose and statistical analysis. USA-WA1/2020 versus BA.1-spike, other sublineage-spikes: 0.053, <0.0001, respective-ly; BA.1-spike versus other sublineage-spike: <0.0001; BA.2-spike versus BA.2.12.1-, BA.3-, BA.4/5-spike: 0.0006, 0.0215, <0.0001, respectively; BA.2.12.1-spike versus BA.3- and BA.4/5-spike: 0.0309, <0.0001, respectively; BA.3-spike versus BA.4/5-spike SARS-CoV-2: <0.0001. (B) FFRNT50 of 2-dose-vaccine-plus-BA.1-infection sera. Thirty-eight sera were collected from individuals who received 3 doses of vaccine and subse-quently contracted BA.1 infection. The p values (Wilcoxon matched-pairs signed-rank test) for group comparison of GMTs are indicated below. USA-WA1/2020 versus all Omicron sublineage-spike: <0.0001; BA.1-spike versus other sublineage-spike: <0.0001; BA.2-spike versus Omicron BA.2.12.1-, BA.3-, BA.4/5, BA.2.75-spike: 0.0082, 0.9095, <0.0001, 0.093, respectively; BA.2.12.1-spike versus Omicron BA.3-, BA.4/5, BA.2.75-spike: 0.0018, <0.0001, 0.58, respectively; BA.3-spike versus BA.4/5-, BA.2.75-spike: <0.0001, 0.062, respectively; BA.4/5-spike versus BA.2.75-spike: <0.0001.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

The explosive spread of the Omicron SARS-CoV-2 variant underscores the importance of analyzing the cross-protection from previous non-Omicron infection. A high-throughput neutralization assay for Omicron SARS-CoV-2 was developed by engineering the Omicron spike gene into an mNeonGreen USA-WA1/2020 SARS-CoV-2. Using this assay, the neutralization titers of patient sera collected were determined at 1-month or 6-months after infection with non-Omicron SARS-CoV-2. From 1-month to 6-month post-infection, the neutralization titers against USA-WA1/2020 decreased from 601 to 142 (a 4.2-fold reduction), while the neutralization titers against Omicron-spike SARS-CoV-2 remained low at 38 and 32, respectively. Thus, at 1-month and 6-months after non-Omicron SARS-CoV-2 infection, the neutralization titers against Omicron were 15.8- and 4.4-fold lower than those against USA-WA1/2020, respectively. The low cross-neutralization against Omicron from previous non-Omicron infection supports vaccination of formerly infected individuals to mitigate the health impact of the ongoing Omicron surge.

The SARS-CoV-2 virus is a betacoronavirus, similar to MERS-CoV and SARS-CoV. All three of these viruses have their origins in bats. The sequences of viruses isolated from U.S. patients are similar to the virus sequences initially posted by China.

One utility of the described reverse genetic system described herein is to facilitate antiviral testing and therapeutic development. The reporter virus allows the use of fluorescence as a surrogate readout for viral replication. Compared with a standard plaque assay or TCID₅₀ quantification, the fluorescent readout shortens the assay turnaround time by several days. In addition, the fluorescent readout offers a quantitative measure that is less labor-intensive than the traditional means of viral titer reduction.

In certain embodiments, a kit can contain nucleic acids and/or expression vectors described herein, as well as transfection and culture reagents. A standard operating procedure (SOP) can provide guidance for use of the kit. The kit system can be used for a variety of research endeavors.

### I. Coronaviruses

Coronaviruses (order Nidovirales, family Coronaviridae) are a diverse group of enveloped, positive-stranded RNA viruses. The coronavirus genome, approximately 27-32 Kb in length, is the largest found in any of the RNA viruses. Large Spike (S) glycoproteins protrude from the virus particle giving coronaviruses a distinctive corona-like appearance when visualized by electron microscopy. Coronaviruses infect a wide variety of species, including canine, feline, porcine, murine, bovine, avian and human (Holmes, et al., 1996, Coronaviridae: the viruses and their replication, p. 1075-1094, Fields Virology, Lippincott-Raven, Philadelphia, Pa.). However, the natural host range of each coronavirus strain is narrow, typically consisting of a single species. Coronaviruses typically bind to target cells through Spike-receptor interactions and enter cells by receptor mediated endocytosis or fusion with the plasma membrane (Holmes, et al., 1996, supra).

Upon entry into susceptible cells, the open reading frame (ORF) nearest the 5′ terminus of the coronavirus genome is translated into a large polyprotein. This polyprotein is autocatalytically cleaved by viral-encoded proteases, to yield multiple proteins that together serve as a virus-specific, RNA-dependent RNA polymerase (RdRP). The RdRP replicates the viral genome and generates 3′ coterminal nested subgenomic RNAs. Subgenomic RNAs include capped, polyadenylated RNAs that serve as mRNAs, and antisense subgenomic RNAs complementary to mRNAs. In one embodiment, each of the subgenomic RNA molecules shares the same short leader sequence fused to the body of each gene at conserved sequence elements known as intergenic sequences (IGS), transcriptional regulating sequences (TRS) or transcription activation sequences. It has been controversial as to whether the nested subgenomic RNAs are generated during positive or negative strand synthesis; however, recent work favors the model of discontinuous transcription during minus strand synthesis (Sawicki, et al., 1995, Adv. Exp. Med. Biol. 380:499-506; Sawicki and Sawicki Adv. Expt. Biol. 1998, 440:215).

A SARS-CoV-2 reference sequence can be found in GenBank accession NC_045512.2 as of March 2, 2020. This sequence is a 29903 bp ss-RNA and is referred to as the Wuhan seafood market pneumonia virus isolate Wuhan-Hu-1. The virus is Severe acute respiratory syndrome coronavirus 2 (SARS-CoV-2) with the taxonomy of Viruses; Riboviria; Nidovirales; Cornidovirineae; Coronaviridae; Orthocoronavirinae; Betacoronavirus; Sarbecovirus. (Wu et al. “A novel coronavirus associated with a respiratory disease in Wuhan of Hubei province, China” Unpublished; NCBI Genome Project, Direct Submission, Submitted (17-JAN-2020) National Center for Biotechnology Information, NIH, Bethesda, MD 20894, USA; Wu et al. Direct Submission, Submitted (05-JAN-2020) Shanghai Public Health Clinical Center and School of Public Health, Fudan University, Shanghai, China).

The genome of SARS-CoV-2 referencing accession NC_045512.2 includes (1) a 5′UTR (1-265), (2) Orflab gene (266-21555), S gene encoding a spike protein (21563..25384), ORF3a gene (25393..26220), E gene encoding E protein (26245..26472), M gene (26523..27191), ORF6 gene (27202..27387), ORF7a gene (27394..27759), ORF7b gene (27756..27887), ORF8 gene (27894..28259), N gene (28274..29533), ORF10 gene (29558..29674), and 3′UTR (29675..29903). In certain aspects, ORF7 (7a and 7b) is substituted by a nucleic acid encoding a reporter protein. Corresponding regions and segment of SEQ ID NO:1 can be determined by alignment with the NC_045512.2 sequence or other similar coronaviruses.

In certain aspects a spike protein can be a spike variant selected from USA-WA1/2020 spike, D614G-spike, XD-spike, Alpha-spike, Belt-spike, Delta-spike, BA.1-spike, BA.2-spike, BA.3-spike, BA.4/5-spike, BA.4/6-spike, BA.2.12.1-spike, BA.2.75-spike, BA.2.75.2-spike, BF7-spike, XBB.1-spike, BQ.1-spike, BQ.1.1-spike, BJ.1-spike, or BA.2.10.4-spike.

The reporter protein is a protein that can be detected, directly or indirectly, and includes colorimetric, fluorescent or luminescent proteins, as well as proteins that bind affinity reagents such as protein/ligand pairs and protein/antibody pairs. Examples of luminescent or marker proteins that can be used in embodiments of the invention include, but are not limited to, Aequorin, firefly luciferase, Renilla luciferase, red luciferase, luxAB, and nanoluciferase. Examples of chemiluminescent protein or marker protein include β-galactosidase, horseradish peroxidase (HRP), and alkaline phosphatase. Examples of fluorescent protein or marker protein include, but are not limited to, mNeonGreen, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOK, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry1, PATagRFP, Kaede (green), Kaede (red), KikGR1 (green), KikGR1 (red), PS-CFP2, PS-CFP2, mEos2 (green), mEos2 (red), PSmOrange, or Dronpa.

### II. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A. Results

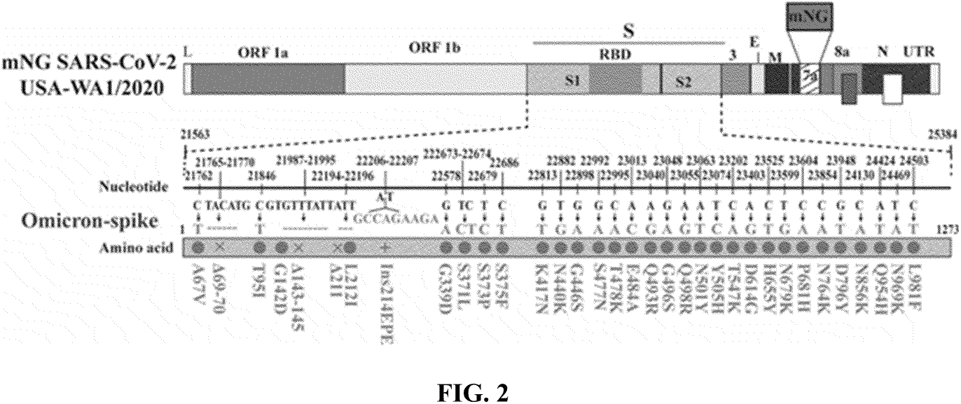

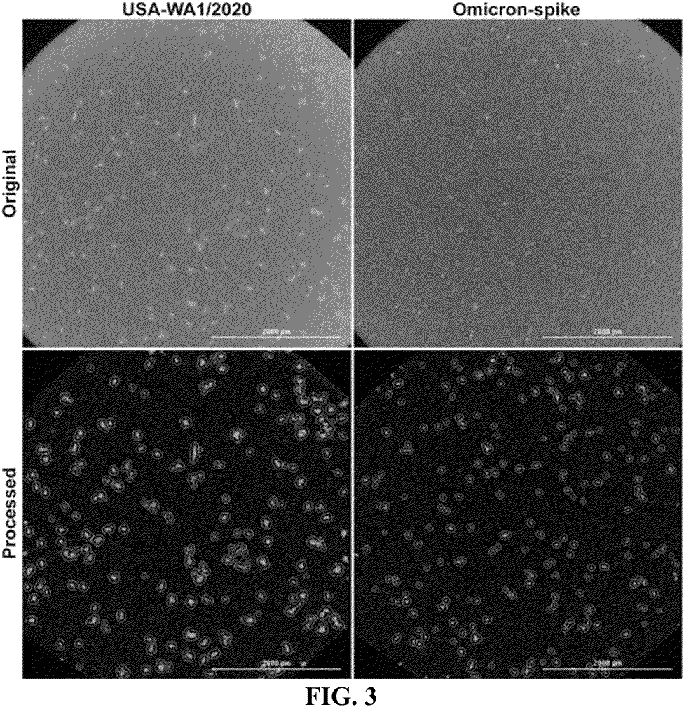

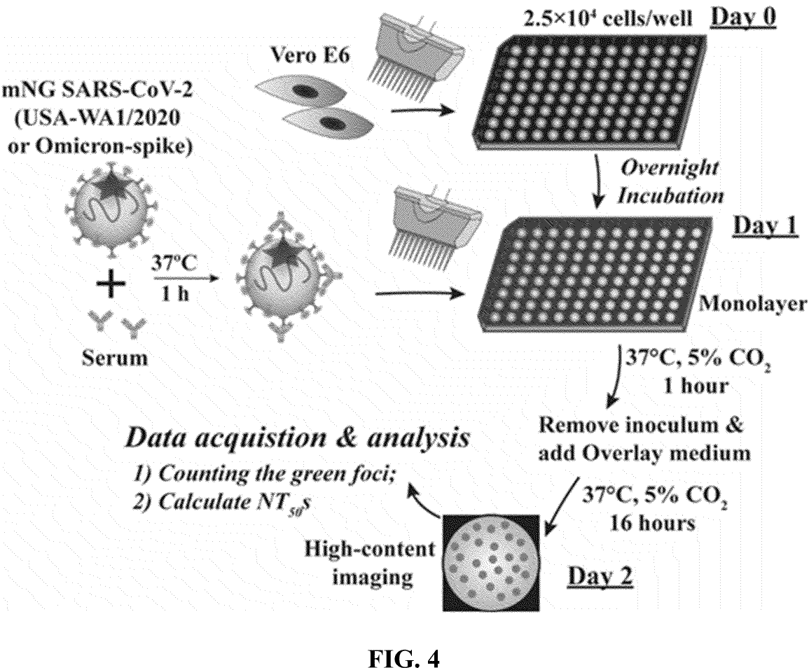

To measure neutralization of the Omicron variant, a high-throughput assay was developed. Using a previously established mNeonGreen (mNG) reporter USA-WA1/2020 SARS-CoV-2, (Muruato et al., Nat Commun 11, 4059, 2020) the original spike gene was swapped with an Omicron spike (BA.1 lineage; GISAID EPI ISL 6640916), resulting in recombinant mNG Omicron-spike SARS-CoV-2 (FIG. 2). The mNG gene was placed at the open-reading-frame-7 (ORF7) of the viral genome (Xie et al. Cell Host Microbe 27, 841-848 e843, 2020). The engineered Omicron spike contained mutations A67V, H69-V70 deletion (D69-70), T95I, G142D, V143-Y145 deletion (D143-145), N211 deletion (D211), L212I, L214 insertion EPE (Ins214EPE), G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F (FIG. 2). The mNG Omicron-spike virus was sequenced to ensure no undesired mutations. After infecting Vero E6 cells, parental mNG USA-WA1/2020 developed larger fluorescent foci than Omicron-spike SARS-CoV-2 (FIG. 3); however, comparable infectious titers of >10⁶ focus-forming units per milliliter (FFU/ml) were obtained for both viruses. The mNG viruses were used to develop a fluorescent focus reduction neutralization test (FFRNT) as depicted in FIG. 4.

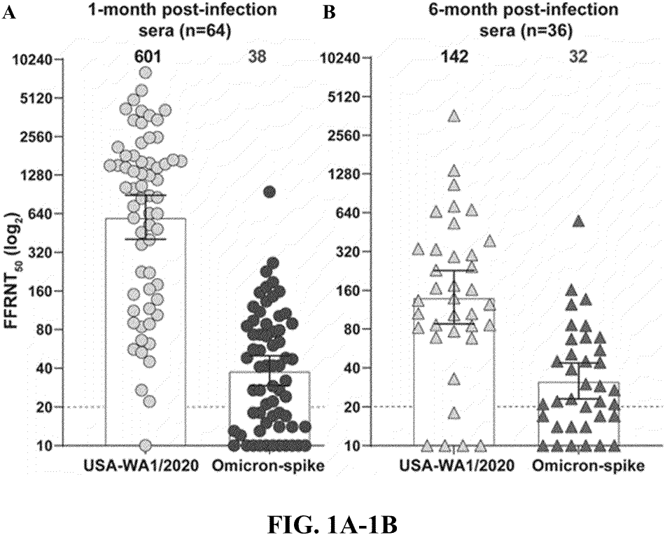

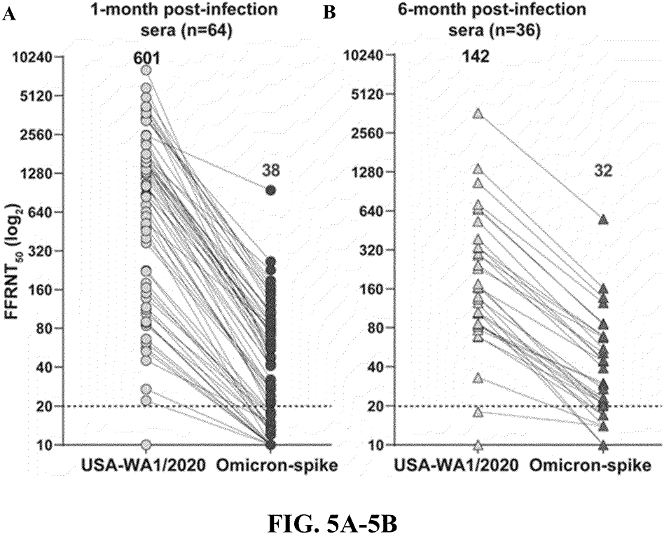

The cross-neutralization of non-Omicron SARS-CoV-2-infected patient sera against Omicron virus was examined. Two panels of COVID-19 patient sera, one collected at 1-month post-infection (n=64) and another collected at 6-month post-infection (N=36), were measured for their 50% fluorescent focus reduction neutralization titers (FFRNT₅₀, defined as the maximal dilution that neutralized 50% of foci) against both USA-WA1/2020 and Omicron-spike SARS-CoV-2. Table 1 and Table 2 summarize the patient information (e.g., age, gender, race, date of positive viral test, symptom, and hospitalization) for the 1-month and 6-month post-infection serum panels. All patients were infected before February 2021, prior to the emergence of the Omicron variant. The 1-month post-infection sera neutralized USA-WA1/2020 and Omicron-spike SARS-CoV-2 with geometric mean titers (GMTs) of 601 and 38, respectively (FIG. 1A and FIG. 5A). Only one serum had a neutralization titer of <20 against USA-WA1/2020, whereas 23 of 64 sera had neutralization titers of <20 against Omicron-spike SARS-CoV-2 (FIG. 1A). Sera with high neutralization titers against USA-WA1/2020 were from symptomatic patients, most were hospitalized (Table 1), confirming that neutralizing antibody levels are associated with COVID-19 disease severity (Gudbjartsson et al., N Engl J Med 383, 1724-34, 2020). Notably, many of the sera with the highest neutralization titers of >3,450 against USA-WA1/2020 were from patients who had received convalescent plasma treatment (Table 1).

The 6-month post-infection sera neutralized USA-WA1/2020 and Omicron-spike SARS-CoV-2 with GMTs of 142 and 32, respectively (FIG. 1B and FIG. 5B). Consistent with the 1-month post-infection results, symptomatic hospitalized patients tended to have higher neutralization titers against USA-WA1/2020 than asymptomatic individuals (Table 2). Thus, from 1-month to 6-months post-infection, the mean neutralization titers against USA-WA1/2020 waned from 601 to 142 (a 4.2-fold decrease), while the neutralization titers against Omicron-spike virus remained low and nearly unchanged at 38 and 32, respectively. Consistent with these results, the waning neutralization overtime against non-Omicron SARS-CoV-2 was previously reported in naturally infected or vaccinated individuals (Falsey et al., N Engl J Med, doi:10.1056/NEJMc2113468, 2021; Chia et al., Lancet Microbe 2, e240-e249, 2021; Widge et al., N Engl J Med 384, 80-82, 2021). The data showed that 1-month and 6-months after non-Omicron SARS-CoV-2 infections, the neutralization titers against Omicron were 15.8- and 4.4-fold lower than those against USA-WA1/2020, respectively. A similar range of neutralization reduction against the Omicron virus was reported for two-dose mRNA-vaccinated sera (Sandile Cele et al., medRxiv, doi:10.1101/2021.12.08.21267417, 2021; Wilhelm et al., MedRiv, doi: 10.1101/2021.12.07.21267432, 2021; Dejnirattisai et al., MedRxiv, doi:10.1101/2021.12.10.21267534, 2021). Collectively, these results demonstrate low cross-neutralization against the Omicron variant from previous non-Omicron viral infection or two-dose mRNA vaccination. The low cross-neutralization against the Omicron variant strongly suggests that individuals previously infected with SARS-CoV-2 should be vaccinated to mitigate Omicron-mediated infection, disease, and potential death.

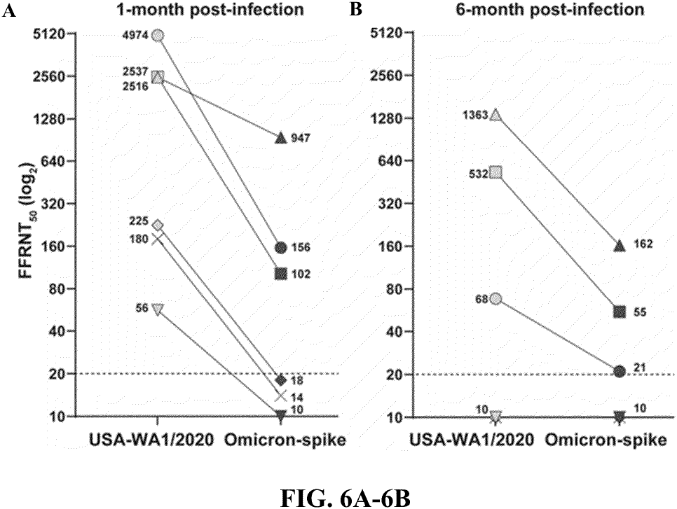

Among all tested sera, only 6 pairs of 1-month and 6-month samples were collected from same individuals (Table 1 and Table 2). Their neutralization patterns (FIG. 6) were similar to those observed with the means from complete 1-month and 6-month serum panels.

The rapid evolution of SARS-CoV-2 underscores the importance of surveillance for new variants and their impact on viral transmission, disease severity, and immune evasion. Surveillance, laboratory investigation, and real-world vaccine effectiveness are essential to guide if and when an Omicron-specific vaccine or booster is needed. Currently, vaccination with booster shots, together with masking and social distance, remain to be the most effective means to mitigate the health impact of Omicron surge. Finally, the high-throughput fluorescent neutralization assay reported in this study can expedite therapeutic antibody screening, neutralization testing, and modified vaccine development.

Table 1

FFRNT₅₀ values of 1-month post-infection sera against mNG USA-WA1/2020 and Omicron-spike SARS-CoV-2

| Serum ID | Age | Gender | Race and Ethnicity | Sample collection date yielding positive viral test | Symptomatic | Hospitalized | FFRNT₅₀ | |
|---|---|---|---|---|---|---|---|---|
| | | | | | | | USA-WA1/2020 | Omicron-spike |
| 1 | 21 | F | Hispanic or Latino | 7/22/2020 | No | No | 10 | 10 |
| 2 | 38 | F | White | 11/27/2020 | No | No | 22 | 10 |
| 3 | 17 | M | Hispanic or Latino | 6/1/2020 | Yes | No | 27 | 10 |
| 4 | 18 | F | Hispanic or Latino | 7/11/2020 | Yes | No | 45 | 15 |
| 5 | 26 | F | Hispanic or Latino | 11/11/2020 | Yes | No | 53 | 10 |
| ▼6 | 24 | F | Hispanic or Latino | 6/24/2020 | No | No | 56 | 10 |
| 7 | 24 | F | Hispanic or Latino | 7/27/2020 | Yes | No | 62 | 10 |
| 8 | 35 | F | Hispanic or Latino | 1/5/2021 | No | No | 66 | 10 |
| 9 | 23 | F | Hispanic or Latino | 6/25/2020 | No | No | 84 | 14 |
| 10 | 24 | F | Hispanic or Latino | 7/2/2020 | Yes | No | 88 | 10 |
| 11 | 33 | F | Black or African American | 11/21/2020 | Yes | No | 90 | 10 |
| 12 | 26 | F | Hispanic or Latino | 7/2/2020 | Yes | No | 104 | 17 |
| 13 | 60 | M | White | 9/26/2020 | Yes | Yes | 112 | 10 |
| 14 | 67 | M | Caucasian/White | 11/16/2020 | Yes | No | 117 | 13 |
| 15 | 22 | M | Hispanic or Latino | 9/24/2020 | No | No | 138 | 18 |
| 16 | 69 | M | White | 12/28/2020 | Yes | No | 151 | 17 |
| 17 | 73 | M | White | 5/27/2020 | Yes | No | 166 | 13 |
| ×18 | 17 | F | Hispanic or Latino | 6/22/2020 | Yes | No | 180 | 14 |
| 19 | 45 | M | Hispanic or Latino | 5/2/2020 | Yes | Yes | 222 | 14 |
| °20 | 24 | F | Hispanic or Latino | 6/24/2020 | Yes | No | 225 | 18 |
| 21 | 25 | F | Black or African American | 7/16/2020 | No | No | 369 | 27 |
| 22 | 75 | F | Hispanic or Latino | 9/14/2020 | Yes | No | 402 | 29 |
| ∗23 | 80 | F | White | 11/9/2020 | Yes | Yes | 459 | 10 |
| 24 | 61 | F | White | 11/3/2020 | Yes | No | 486 | 27 |
| 25 | 78 | M | White | 12/15/2020 | Yes | No | 524 | 85 |
| 26 | 66 | M | White | 5/30/2020 | Yes | Yes | 592 | 12 |
| 27 | 38 | M | Hispanic or Latino | 6/27/2020 | No | No | 640 | 24 |
| 28 | 34 | M | Hispanic or Latino | 11/11/2020 | Yes | No | 645 | 22 |
| 29 | 25 | F | Hispanic or Latino | 7/17/2020 | No | No | 730 | 21 |
| 30 | 66 | M | Black or African American | 4/27/2020 | Yes | Yes | 840 | 42 |
| 31 | 39 | M | Black or African American | 4/9/2020 | Yes | Yes | 856 | 27 |
| 32 | 35 | F | Hispanic or Latino | 10/15/2020 | Yes | Yes | 882 | 77 |
| 33 | 55 | M | Hispanic or Latino | 5/6/2020 | Yes | Yes | 1003 | 89 |
| ∗34 | 67 | F | Hispanic or Latino | 1/4/2021 | Yes | Yes | 1020 | 41 |
| 35 | 55 | F | White | 9/28/2020 | Yes | No | 1050 | 32 |
| 36 | 40 | F | Hispanic or Latino | 12/20/2020 | Yes | No | 1174 | 55 |
| 37 | 60 | F | Hispanic or Latino | 11/27/2020 | Yes | Yes | 1268 | 48 |
| 38 | 65 | M | Hispanic or Latino | 5/7/2020 | Yes | Yes | 1306 | 64 |
| ∗39 | 69 | M | White | 11/22/2020 | Yes | Yes | 1365 | 79 |
| 40 | 68 | M | Caucasian/White | 5/10/2020 | Yes | Yes | 1454 | 73 |
| 41 | 50 | M | Black or African American | 4/8/2020 | Yes | Yes | 1465 | 60 |
| 42 | 63 | M | Hispanic or Latino | 1/23/2021 | Yes | Yes | 1517 | 94 |
| 43 | 39 | M | Black or African American | 3/31/2020 | Yes | Yes | 1519 | 110 |
| 44 | 72 | M | White | 12/23/2020 | Yes | Yes | 1555 | 73 |
| 45 | 55 | F | White | 10/6/2020 | Yes | Yes | 1584 | 107 |
| 46 | 57 | M | White | 7/5/2020 | Yes | No | 1618 | 18 |
| 47 | 1 | F | Hispanic or Latino | 1/18/2021 | Yes | Yes | 1638 | 227 |
| 48 | 87 | M | White | 1/5/2021 | Yes | Yes | 1679 | 71 |
| 49 | 96 | F | White | 12/30/2020 | Yes | Yes | 1807 | 88 |
| 50 | 66 | M | Hispanic or Latino | 12/19/2020 | Yes | No | 1814 | 159 |
| 51 | 75 | M | Hispanic or Latino | 10/27/2020 | Yes | No | 2119 | 56 |
| 52 | 63 | F | Hispanic or Latino | 12/12/2020 | Yes | Yes | 2289 | 42 |
| △53 | 66 | M | White | 12/27/2020 | Yes | Yes | 2516 | 947 |
| □54 | 49 | M | Black or African American | 1/3/2021 | No | Yes | 2537 | 102 |
| 55 | 56 | M | Hispanic or Latino | 7/13/2020 | Yes | Yes | 3277 | 265 |
| 56 | 44 | F | Black or African American | 8/20/20/ | Yes | Yes | 3443 | 133 |
| ∗57 | 83 | M | Hispanic or Latino | 11/22/2020 | Yes | Yes | 3464 | 188 |
| ∗58 | 75 | M | White | 12/27/2020 | Yes | Yes | 3741 | 172 |
| ∗59 | 74 | M | White | 12/21/2020 | Yes | Yes | 4055 | 42 |
| 60 | 48 | F | Hispanic or Latino | 6/21/2020 | Yes | Yes | 4116 | 121 |
| 61 | 78 | F | Hispanic or Latino | 12/16/2020 | Yes | Yes | 4216 | 146 |
| °∗62 | 70 | M | Hispanic or Latino | 12/12/2020 | Yes | Yes | 4974 | 156 |
| ∗63 | 49 | M | White | 12/29/2020 | Yes | Yes | 5876 | 47 |
| 64 | 50 | F | Hispanic or Latino | 11/9/2020 | Yes | Yes | 8088 | 48 |
| GMT | 44 | - | - | - | - | - | 601 | 38 |
| 95%CI | 37-52 | - | - | - | - | - | 405-891 | 29-50 |

∗Patients received convalescent plasma treatment.
∇×◊△□∘ Patients who gave both 1- and 6-month post-infection sera.

TABLE 2

FFRNT₅₀ values of 6-month post-infection sera against mNG USA-WA1/2020 and Omicron-spike SARS-CoV-2

| Serum ID | Age | Gender | Race and Ethnicity | Sample collection date yielding positive viral test | Symptomatic | Hospitalized | FFRNT₅₀ | |
|---|---|---|---|---|---|---|---|---|
| | | | | | | | USA-WA1/2020 | Omicron-spike |
| ▼1 | 17 | F | Hispanic or Latino | 6/22/2020 | Yes | No | 10 | 10 |
| ×2 | 24 | F | Hispanic or Latino | 6/24/2020 | No | No | 10 | 10 |
| ◊3 | 24 | F | Hispanic or Latino | 6/24/2020 | Yes | No | 10 | 10 |
| 4 | 70 | M | White | 7/26/2020 | No | No | 10 | 10 |
| 5 | 29 | F | Black or African American | 8/3/2020 | No | No | 18 | 14 |
| 6 | 21 | F | Hispanic or Latino | 6/26/2020 | No | No | 33 | 14 |
| ◦∗7 | 70 | M | Hispanic or Latino | 12/12/2020 | Yes | Yes | 68 | 21 |
| 8 | 27 | F | Hispanic or Latino | 8/9/2020 | No | No | 69 | 17 |
| 9 | 22 | F | Hispanic or Latino | 10/1/2020 | No | No | 77 | 30 |
| 10 | 61 | F | White | 8/24/2020 | No | No | 82 | 29 |
| 11 | 40 | F | Hispanic or Latino | 7/31/2020 | Yes | No | 85 | 21 |
| 12 | 22 | F | Hispanic or Latino | 7/13/2020 | No | No | 86 | 22 |
| 13 | 50 | F | White | 11/25/2020 | Yes | Yes | 86 | 17 |
| 14 | 26 | F | Hispanic or Latino | 9/10/2020 | No | No | 103 | 22 |
| 15 | 21 | F | Hispanic or Latino | 6/2/2020 | Yes | No | 105 | 10 |
| 16 | 26 | F | Hispanic or Latino | 9/10/2020 | No | No | 106 | 23 |
| 17 | 73 | F | White | 10/14/2020 | Yes | Yes | 125 | 10 |
| 18 | 22 | M | Hispanic or Latino | 9/24/2020 | Yes | Yes | 134 | 27 |
| 19 | 47 | M | Black or African American | 4/23/2020 | No | No | 140 | 14 |
| 20 | 79 | M | White | 5/4/2020 | Yes | Yes | 163 | 44 |
| ∗∗21 | 77 | F | Black or African American | 12/7/2020 | Yes | No | 167 | 20 |
| 22 | 57 | M | White | 5/13/2020 | Yes | Yes | 175 | 17 |
| 23 | 23 | F | Hispanic or Latino | 12/25/2020 | Yes | No | 230 | 67 |
| 24 | 40 | F | Hispanic or Latino | 3/16/2020 | Yes | No | 244 | 51 |
| 25 | 22 | F | Hispanic or Latino | 8/11/2020 | Yes | No | 292 | 69 |
| 26 | 54 | M | White | 4/10/2020 | Yes | Yes | 302 | 39 |
| 27 | 64 | M | White | 1/3/2021 | Yes | Yes | 333 | 69 |
| 28 | 39 | F | Black or African American | 7/9/2020 | Yes | No | 337 | 45 |
| 29 | 69 | M | White | 8/14/2020 | Yes | No | 389 | 45 |
| □30 | 49 | M | Black or African American | 1/3/2021 | Yes | Yes | 532 | 55 |
| 31 | 96 | F | White | 12/30/2020 | Yes | Yes | 655 | 86 |
| 32 | 80 | F | Hispanic or Latino | 6/20/2020 | Yes | No | 675 | 85 |
| 33 | 49 | F | Hispanic or Latino | 3/26/2020 | Yes | No | 719 | 125 |
| 34 | 48 | F | Hispanic or Latino | 6/21/2020 | Yes | Yes | 1059 | 137 |
| △35 | 66 | M | White | 12/27/2020 | Yes | Yes | 1363 | 162 |
| 36 | 70 | M | Hispanic or Latino | 11/19/2020 | Yes | Yes | 3648 | 554 |
| GMT | 41 | - | - | - | - | - | 142 | 32 |
| 95% Cl | 35-49 | - | - | - | - | - | 88-229 | 23-44 |

∗Patients received convalescent plasma treatment.
∗∗Patient received therapeutic antibody treatment.
∇×◊△□∘ Patients who gave both 1- and 6-month post-infection sera.

### B. Methods

Construction of recombinant viruses. The recombinant mNeoGreen (mNG) Omicron-spike SARS-CoV-2 was constructed on the genetic background of an infectious cDNA clone derived from clinical strain WA1 (2019-nCoV/USA_WA1/2020) containing an mNG reporter gene (Xie et al., Cell Host Microbe 27, 841-848 e843, 2020). The Omicron spike mutations, including A67V, Δ69-70, T95I, G142D, Δ143-145, Δ211, L212I, Ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F, were engineered using a PCR-based mutagenesis protocol as reported previously (Plante et al., Nature, doi:10.1038/s41586-020-2895-3, 2020). The full-length genomic cDNAs were in vitro ligated and used as templates to transcribe full-length viral RNA. Mutant viruses were recovered on day 3 after Vero E6 cells were electroporated with the in vitro RNA transcripts. The harvested virus stocks were quantified for their infectious titers (fluorescent focus units) by titrating the viruseson Vero E6 cells in a 96-well plate after 16 h of infection. The genome sequences of the virus stocks were confirmed to have no undesired mutations by Sanger sequencing. The detailed protocol of genome sequencing was recently reported (Xie et al., Nature Protocols 16, 1761-1784, doi:10.1038/s41596-021-00491-8, 2021).

Serum specimens. The research protocol regarding the use of human serum specimens was reviewed and approved by the University of Texas Medical Branch (UTMB) Institutional Review Board (IRB#: 20-0070). The de-identified convalescent sera from COVID-19 patients (confirmed by the molecular tests with FDA’s Emergency Use Authorization) were heat-inactivated at 56°C for 30 min before testing.

Fluorescent focus reduction neutralization test. Neutralization titers of human sera were measured by a fluorescent focus reduction neutralization test (FFRNT) using the mNG reporter SARS-CoV-2. Briefly, Vero E6 cells (2.5 × 10⁴) were seeded in each well of black µCLEAR flat-bottom 96-well plate (Greiner Bio-one™). The cells were incubated overnight at 37°C with 5% CO₂. On the following day, each serum was 2-fold serially diluted in the culture medium with the first dilution of 1:20. The diluted serum was incubated with 100-150 fluorescent focus units (FFU) of mNG SARS-CoV-2 at 37°C for 1 h (final dilution range of 1:20 to 1:20,480), after which the serum-virus mixtures were inoculated onto the pre-seeded Vero E6 cell monolayer in 96-well plates. After 1 h infection, the inoculum was removed and 100 µl of overlay medium (DMEM supplemented with 0.8% methylcellulose, 2% FBS, and 1% P/S) was added to each well. After incubating the plates at 37°C for 16 h, raw images of mNG fluorescent foci were acquired using Cytation™ 7 (BioTek) armed with 2.5× objective and processed using the default software setting. The foci in each well were counted and normalized to the non-serum-treated controls to calculate the relative infectivities. The curves of the relative infectivity versus the serum dilutions (log₁₀ values) were plotted using Prism 9 (GraphPad). A nonlinear regression method was used to determine the dilution fold that neutralized 50% of mNG SARS-CoV-2 (defined as FFRNT₅₀). Each serum was tested in duplicates.

Statistics - The nonparametric Wilcoxon matched-pairs signed rank test was used to analyze the statistical significance in FIG. 1.

### Example 2

### A. Results and Discussions

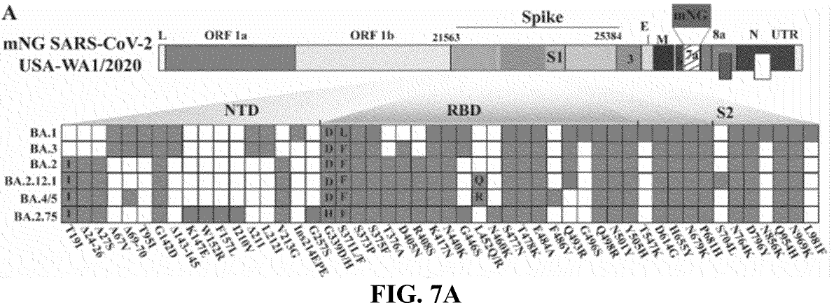

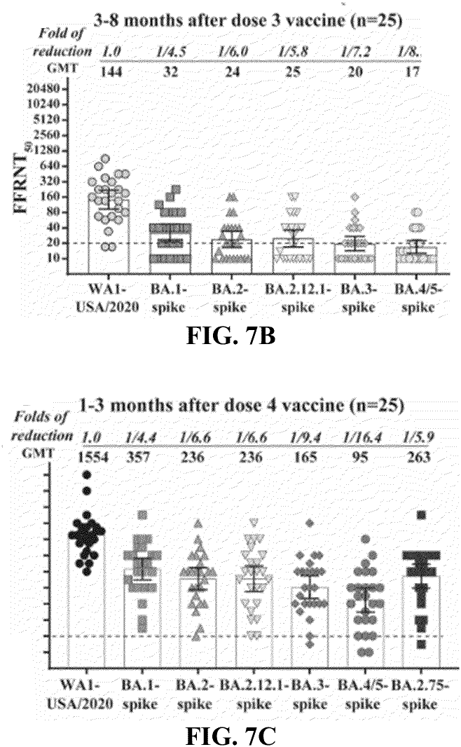

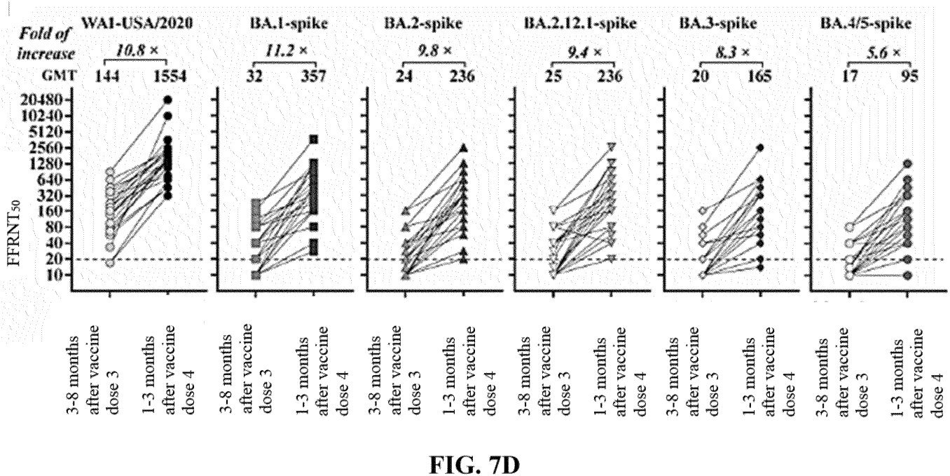

Experimental approach and rationale. A set of previously established recombinant SARS-CoV-2s were used to determine the serum neutralization against different Omicron sublineages. Each recombinant SARS-CoV-2 contained a complete spike gene from BA.1, BA.2, BA.2.12.1, BA.3, or BA.4/5 in the backbone of USA-WA1/2020 (a virus strain isolated in January 2020) containing an mNeonGreen (mNG) reporter, resulting in BA.1-, BA.2-, BA.2.12.1-, BA3-, or BA.4/5-spike mNG SARS-CoV-2 (Kurhade et al., 2022b). BA.4 and BA.5 have an identical spike sequence and are denoted as BA.4/5. FIG. 7A summarizes the amino acid mutations of the spike protein from different Omicron sublineages. An mNeonGreen (mNG) gene was engineered into the open-reading-frame-7 (ORF7) of the viral genome to enable a fluorescent focus reduction neutralization test (FFRNT) in a high-throughput format (Zou et al., 2022a). The insertion of mNG reporter anntenuated SARS-CoV-2 replication and pathohgenesis (Johnson et al., 2022; Liu et al., 2022).The FFRNT has been reliably used to measure antibody neutralization for COVID-19 vaccine research and development (Kurhade et al., 2022a; Kurhade et al., 2022b).

Using FFRNT, the neutralization of three panels of human sera were measured against the chimeric Omicron sublineage-spike mNG SARS-CoV-2s. The first panel consisted of 25 pairs of sera collected from individuals before and after dose 4 of Pfizer or Moderna’s original vaccine (Table 3). Those specimens were tested negative against viral nucleocapsid protein, suggesting those individuals had not been infected by SARS-CoV-2. The second and third serum panels were collected from individuals who had received 2 (n=29; Table 4) or 3 (n=38; Table 5) doses of the original mRNA vaccine and subsequently contracted Omicron BA.1 breakthrough infection. The BA.1 breakthrough infection was confirmed for each patient by sequencing viral RNA collected from nasopharyngeal swab samples. Tables 3-5 summarize (i) the serum information and (ii) the 50% fluorescent focus-reduction neutralization titers (FFRNT₅₀) against USA-WA1/2020, BA.1-, BA.2-, BA.2.12.1-, BA.3-, and BA.4/5-spike SARS-CoV-2s. The description and analysis of the FFRNT₅₀ results against different Omicron sublineages are detailed in the following sections for each serum panel.

The booster effect by dose 4 mRNA vaccine is less pronounced against BA.4/5 compared to USA-WA1/2020 and other omicron sublineages. To measure 4 doses of vaccine-elicited neutralization, 25 pairs of sera were collected from individuals before and after dose 4 of Pfizer or Moderna mRNA vaccine. For each serum pair, one sample was collected 3-8 months after dose 3 vaccine; the other sample was obtained from the same individual 1-3 months after dose 4 vaccine (Table 3). Before the 4^{th} dose vaccine, the 3-dose-vaccine sera neutralized USA-WA1/2020, BA.1-, BA.2-, BA.2.12.1-, BA.3-, and BA.4/5-spike mNG viruses with low geometric mean titers (GMTs) of 144, 32, 24, 25, 20, and 17, respectively (FIG. 7B); after the 4^{th} dose vaccine, the GMTs increased significantly to 1554, 357, 236, 236, 165, and 95, respectively (FIG. 7C); so, the 4^{th} dose vaccine significantly increased the neutralization against the corresponding viruses by 10.8-, 11.2-, 9.8-, 9.4-, 8.3-, and 5.6-fold, respectively (FIG. 7D). Despite the significant increase in neutralization after the 4^{th} dose vaccine, the GMTs against BA.1-, BA2-, BA2.12.1-, BA.3-, and BA.4/5-spike viruses were 4.4-, 6.6-, 6.6-, 9.4-, and 16.4-fold lower than the GMT against the USA-WA1/2020, respectively (FIG. 7C). These results support three conclusions. First, among the tested Omicron sublineages, BA.5 possesses the greatest evasion of vaccine-elicited neutralization. The results are in agreement with other studies supporting that BA.5 and other Omicron sublineages efficiently evade vaccine-elicited neutralization (Arora et al., 2022; Cao et al., 2022; Hachmann et al., 2022; Sheward et al., 2022; Tan et al., 2022). Second, the booster effect by the 4^{th} dose is less pronounced against BA.4/5 compared to USA-WA1/2020 and other omicron sublineages. It should be noted that dose 4 did increase the neutralizing GMT against BA.4/5 from 17 (FIG. 7B) to 95 (FIG. 7C). A recent study reported a neutralizing titer of 70 as the threshold to prevent breakthrough infections of Delta variant (Zou et al., 2022b). Although the minimal neutralizing titer required to prevent BA.5 infection has not been determined, the low neutralization against BA.5 after dose 3 vaccine [GMT of 103 at 1-month post dose 3, reported by Kurhade et al. (Kurhade et al., 2022b)] and dose 4 vaccine (GMT of 95 at 1- to 3-month post dose 4, reported here), together with the increased viral transmissibility, could account for the ongoing surge of BA.5 around the world. Third, an updated vaccine that matches the highly immune-evasive and prevalent BA.5 spike is needed to mitigate the current and future Omicron surges. The results support the U.S. Food and Drug Administration’s recommendation to include BA.5 spike for future COVID-19 vaccine booster doses.

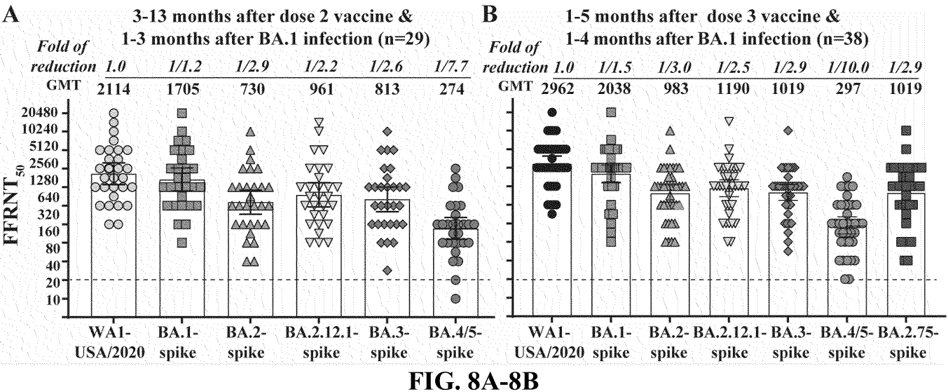

High neutralization against BA.5 and other Omicron sublineages after 2 or 3 doses of vaccine plus BA.1 infection. To compare with 4-dose-vaccine sera, we measured the neutralization against Omicron sublineages using sera collected from individuals who had received 2 or 3 doses of the original mRNA vaccine and subsequently contracted BA.1 infection (FIG. 8). Tables 4 and 5 summarize the FFRNT₅₀ results for 2-dose-vaccine-plus-BA.1-infection sera and 3-dose-vaccine-plus-BA.1-infection sera, respectively. The 2-dose-vaccine-plus-BA.1-infection sera neutralized BA.1, BA.2, BA.2.12.1, BA.3, and BA.4/5 with GMTs of 2114, 1705, 730, 961, 813, and 274, respectively (FIG. 8A); the 3-dose-vaccine-plus-BA.1-infection sera showed slightly higher GMTs of 2962, 2038, 983, 1190, 1019, and 297, respectively (FIG. 8B). So, the GMT ratios between the 3-dose-vaccine-plus-BA.1-infection sera and 2-dose-vaccine-plus-BA.1-infection sera were 1.4, 1.2, 1.3, 1.2, 1.3, and 1.1 when neutralizing USA-WA1/2020, BA.1-, BA.2-, BA.2.12.1-, BA.3-, and BA.4/5-spike viruses, respectively; these GMT differences between the two serum groups were statistically insignificant, suggesting the extra dose of vaccine does not significantly boost neutralization for the 3-dose-vaccine-plus-BA.1-infection sera.

In contrast, the GMT ratios between the 2-dose-vaccine-plus-BA.1-infection and 4-dose-vaccine sera were 1.4, 4.8, 3.1, 4.1, 4.9, and 3.9 when neutralizing USA-WA1/2020, BA.1-, BA.2-, BA.2.12.1-, BA.3-, and BA.4/5-spike viruses, respectively. The result suggests that, compared with the two extra doses of vaccine in the 4-dose-vaccine sera, the BA.1 infection in the 2-dose-vaccine-plus-BA.1-infection sera is more efficient in boosting both the magnitude and breadth of neutralization against all Omicron sublineages; however, the neutralization against BA.5 was still the lowest among all tested sublineages.

For the 2-dose-vaccine-plus-BA.1-infection sera, the GMTs against BA.1-, BA.2-, BA.2.12.1-, BA.3-, and BA.4/5-spike viruses were 1.2-, 2.9-, 2.2-, 2.6-, and 7.7-fold lower than the GMT against the USA-WA1/2020, respectively (FIG. 8A); similar results were observed for the 3-dose-vaccine-plus-BA.1-infection sera, with GMTs against BA.1-, BA.2-, BA.2.12.1-, BA.3-, and BA.4/5-spike viruses that were 1.5-, 3.0-, 2.5-, 2.9-, and 10-fold lower than the GMT against the USA-WA1/2020, respectively (FIG. 8B). The GMT decreases against Omicron sublineages for the 2-dose-vaccine-plus-BA.1-infection sera and those for the 3-dose-vaccine-plus-BA.1-infection sera are significantly less than those observed for the 4-dose-vaccine sera (Compare FIGS. 8A and 8B with FIG. 7C). The results again indicate that BA.1 infection of vaccinated people efficiently boosts the breadth of neutralization against all tested Omicron sublineages. However, such BA.1 infection-mediated boost of neutralizing magnitude/breadth is dependent on previous vaccination. This is because BA.1 infection of unvaccinated people did not elicit greater neutralizing magnitude/breadth against Omicron sublineages than 3 doses of mRNA vaccine (Kurhade et al., 2022b).

Neutralization against Omicron sublineage BA.2.75. To assess the neutralization of the newly emerged Omicron sublineage BA.2.75, the complete spike gene of BA.2.75 (FIG. 7A) was engineered into the backbone of mNG USA-WA1/2020, resulting in BA.2.75-spike mNG SARS-CoV-2. The BA.2.75-spike mNG SARS-CoV-2 was sequenced to ensure no undesired mutations. When tested with 4-dose-vaccine sera, the neutralizing GMT against BA.2.75-spike virus was 2.8-fold higher than that against BA.5-spike virus (FIG. 7C). Similarly, when tested with 3-dose-vaccine-plus-BA.1-infection sera, the neutralizing GMT against BA.2.75-spike virus was 3.4-fold higher than that against BA.5-spike virus (FIG. 8B). Collectively, the results indicate that BA.2.75 is less immune-evasive than BA.5.

TABLE 3

Twenty-five pairs of human serum samples collected after dose 3 and 4 of mRNA vaccine, Related to FIG. 7

| Serum ID | Pair # | Age (year) | Gender (F/M) | Ethnicity | Serum collection time (days post-dose 3 vaccination) | Serum collection time (days post-dose 4 vaccination) | Interval days between dose 3 & 4 vaccination | mRNA Vaccine type | ∗FFRNT₅₀ | | | | | | |
|---|---|---|---|---|---|---|---|---|---|---|---|---|---|---|---|
| | | | | | | | | | USA-WA1/2020 | BA.1-spike | BA.2-spike | BA.2.12. 1-spike | BA.3-spike | BA.4/5-spike | BA.2.75-spike |
| 1 | 1 | 62 | F | White | 174 | | 174 | Pfizer | 17 | ^10 | 10 | 10 | 10 | 10 | - |
| 2 | | | | | | 37 | | | 320 | 40 | 28 | 20 | 20 | 10 | 40 |
| 3 | 2 | 84 | M | White | 186 | | 217 | Pfizer | 17 | 10 | 10 | 10 | 10 | 10 | - |
| 4 | | | | | | 26 | | | 640 | 160 | 80 | 80 | 80 | 20 | 80 |
| 5 | 3 | 80 | M | Hispanic | 184 | | 196 | Pfizer | 34 | 10 | 10 | 10 | 10 | 10 | - |
| 6 | | | | | | 30 | | | 10240 | 453 | 640 | 905 | 320 | 320 | 640 |
| 7 | 4 | 78 | M | White | 184 | | 209 | Pfizer | 57 | 10 | 10 | 10 | 10 | 10 | - |
| 8 | | | | | | 56 | | | 453 | 28 | 20 | 20 | 14 | 10 | 14 |
| 9 | 5 | 87 | M | White | 243 | | 247 | Pfizer | 57 | 10 | 10 | 10 | 10 | 10 | - |
| 10 | | | | | | 24 | | | 1810 | 1280 | 453 | 640 | 453 | 113 | 640 |
| 11 | 6 | 66 | F | White | 184 | | 241 | Pfizer | 67 | 20 | 10 | 10 | 10 | 10 | - |
| 12 | | | | | | 48 | | | 453 | 160 | 80 | 40 | 40 | 20 | 28 |
| 13 | 7 | 83 | F | White | 148 | | 222 | Pfizer | 67 | 10 | 10 | 10 | 10 | 10 | - |
| 14 | | | | | | 23 | | | 1522 | 320 | 320 | 320 | 160 | 160 | 320 |
| 15 | 8 | 84 | F | White | 174 | | 209 | Pfizer | 80 | 10 | 20 | 14 | 10 | 10 | - |
| 16 | | | | | | 55 | | | 1076 | 160 | 226 | 160 | 113 | 80 | 160 |
| 17 | 9 | 86 | M | White | 189 | | 215 | Pfizer | 95 | 40 | 28 | 40 | 20 | 10 | - |
| 18 | | | | | | 51 | | | 1522 | 640 | 320 | 320 | 320 | 20 | 640 |
| 19 | 10 | 87 | F | Hispanic | 195 | | 259 | Pfizer | 135 | 40 | 20 | 40 | 20 | 20 | - |
| 20 | | | | | | 43 | | | 1076 | 320 | 320 | 226 | 160 | 160 | 113 |
| 21 | 11 | 67 | F | Black | 186 | | 233 | Pfizer | 135 | 20 | 10 | 10 | 10 | 10 | - |
| 22 | | | | | | 50 | | | 1522 | 160 | 160 | 226 | 80 | 80 | 160 |
| 23 | 12 | 86 | M | White | 156 | | 217 | Pfizer | 135 | 40 | 40 | 80 | 20 | 40 | - |
| 24 | | | | | | 49 | | | 1810 | 640 | 320 | 320 | 160 | 320 | 320 |
| 25 | 13 | 80 | M | Black | 184 | | 240 | Pfizer | 160 | 10 | 10 | 10 | 10 | 10 | - |
| 26 | | | | | | 44 | | | 1280 | 640 | 320 | 453 | 320 | 160 | 320 |
| 27 | 14 | 72 | F | White | 191 | | 230 | Pfizer | 190 | 20 | 14 | 10 | 10 | 10 | - |
| 28 | | | | | | 52 | | | 1522 | 1280 | 320 | 453 | 320 | 40 | 453 |
| 29 | 15 | 75 | F | White | 143 | | 209 | Pfizer | 190 | 40 | 28 | 28 | 20 | 14 | - |
| 30 | | | | | | 78 | | | 2560 | 905 | 905 | 1280 | 640 | 453 | 640 |
| 31 | 16 | 73 | M | White | 163 | | 192 | Moderna | 226 | 40 | 20 | 80 | 40 | 40 | - |
| 32 | | | | | | 94 | | | 640 | 80 | 57 | 40 | 57 | 40 | 40 |
| 33 | 17 | 75 | M | Black | 198 | | 207 | Pfizer | 226 | 80 | 40 | 40 | 40 | 40 | - |
| 34 | | | | | | 47 | | | 2153 | 453 | 113 | 57 | 80 | 57 | 640 |
| 35 | 18 | 80 | M | White | 146 | | 205 | Pfizer | 226 | 80 | 80 | 20 | 20 | 14 | - |
| 36 | | | | | | 52 | | | 3620 | 1280 | 1280 | 1280 | 640 | 640 | 640 |
| 37 | 19 | 78 | M | White | 196 | | 214 | Pfizer | 320 | 80 | 40 | 40 | 40 | 20 | - |
| 38 | | | | | | 73 | | | 761 | 226 | 80 | 80 | 80 | 40 | 320 |
| 39 | 20 | 92 | F | White | 163 | | 228 | Pfizer | 320 | 113 | 80 | 80 | 40 | 20 | - |
| 40 | | | | | | 27 | | | 2153 | 160 | 160 | 160 | 80 | 113 | 640 |
| 41 | 21 | 59 | F | Hispanic | 203 | | 254 | Pfizer (dose 1-3) Moderna (dose 4) | 381 | 40 | 20 | 20 | 20 | 10 | - |
| 42 | | | | | | 27 | | | 1810 | 640 | 640 | 1280 | 320 | 160 | 453 |
| 43 | 22 | 90 | M | Black | 231 | | 231 | Pfizer | 453 | 80 | 40 | 40 | 57 | 20 | - |
| 44 | | | | | | 34 | | | 2560 | 453 | 320 | 320 | 320 | 160 | 320 |
| 45 | 23 | 94 | F | White | 110 | | 228 | Pfizer | 453 | 40 | 40 | 40 | 20 | 40 | - |
| 46 | | | | | | 47 | | | 1280 | 320 | 160 | 160 | 80 | 80 | 320 |
| 47 | 24 | 71 | F | White | 143 | | 243 | Moderna (dose 1-3) Pfizer (dose 4) | 640 | 226 | 160 | 160 | 160 | 80 | - |
| 48 | | | | | | 27 | | | 2153 | 905 | 640 | 640 | 640 | 320 | 640 |
| 49 | 25 | 84 | F | White | 235 | | 257 | Pfizer | 905 | 160 | 160 | 160 | 80 | 80 | - |
| 50 | | | | | | 35 | | | 20480 | 3620 | 2560 | 2560 | 2560 | 1280 | 3620 |
| #GMT PD3 | - | - | - | - | - | - | - | - | 144 | 32 | 24 | 25 | 20 | 17 | - |
| †95% Cl PD3 | | | | | | | | | 93-220 | 22-48 | 17-34 | 17-37 | 14-27 | 13-23 | - |
| GMT PD4 | - | - | - | - | - | - | - | - | 1554 | 357 | 236 | 236 | 165 | 95 | 263 |
| 95% Cl PD4 | | | | | | | | | 1069-2261 | 224-569 | 147-379 | 136-409 | 101-268 | 56-160 | 157-441 |

∗Individual FFRNT₅₀ value is the geometric mean of duplicate plaque assay results.
^FFRNT₅₀ of <20 was treated as 10 for plot purposes and statistical analysis.
#Geometric mean neutralizing titers (GMT).
†95% confidence interval (95% CI) for the GMT.

TABLE 4

Twenty-nine human serum samples collected after 2 doses of mRNA vaccine and a subsequent Omicron BA.1 breakthrough infection, Related to FIG. 8

| Sample ID | Age (year) | Gender (M/F) | Ethnicity | Vaccine type | COVID test positive days post last vaccine | Serum collection time (Post COVID test positive days) | ∗FFRNT₅₀ | | | | | |
|---|---|---|---|---|---|---|---|---|---|---|---|---|
| | | | | | | | USA-WA1/2020 | BA.1-spike | BA.2-spike | BA.2.12.1-spike | BA.3-spike | BA.4/5-spike |
| 1 | 37 | F | White | Moderna | 285 | 59 | 320 | 160 | 80 | 160 | 57 | 20 |
| 2 | 32 | M | Hispanic | Pfizer | 187 | 73 | 320 | 320 | 80 | 160 | 160 | 80 |
| 3 | 20 | M | Hispanic | Moderna | 401 | 48 | 640 | 640 | 320 | 320 | 320 | 160 |
| 4 | 96 | F | Hispanic | Pfizer | 341 | 50 | 640 | 640 | 226 | 320 | 320 | 113 |
| 5 | 87 | M | White | Moderna | 350 | 51 | 640 | 640 | 160 | 226 | 160 | 40 |
| 6 | 36 | F | White | Pfizer | 191 | 71 | 640 | 320 | 160 | 160 | 160 | 80 |
| 7 | 31 | F | Black | Pfizer | 124 | 58 | 905 | 640 | 320 | 320 | 320 | 320 |
| 8 | 51 | F | White | Moderna | 165 | 15 | 1280 | 1280 | 640 | 905 | 640 | 226 |
| 9 | 57 | M | Asian | Pfizer | 392 | 30 | 1280 | 1280 | 320 | 905 | 640 | 160 |
| 10 | 78 | M | Black | Moderna | 152 | 34 | 1280 | 1810 | 905 | 1280 | 1280 | 320 |
| 11 | 64 | F | Black | Pfizer | 232 | 35 | 1280 | 1280 | 640 | 905 | 640 | 320 |
| 12 | 30 | M | Asian | Pfizer | 214 | 64 | 1280 | 905 | 320 | 453 | 320 | 160 |
| 13 | 74 | F | White | Pfizer | 389 | 34 | 1810 | 1280 | 320 | 453 | 320 | 160 |
| 14 | 21 | F | Hispanic | Moderna | 254 | 49 | 1810 | 2560 | 1280 | 1280 | 1280 | 226 |
| 15 | 31 | F | White | Pfizer | 317 | 105 | 1810 | 1280 | 640 | 640 | 640 | 320 |
| 16 | 62 | M | White | Pfizer | 316 | 29 | 2560 | 1810 | 640 | 640 | 1280 | 320 |
| 17 | 38 | F | Asian | Pfizer | 98 | 33 | 2560 | 2560 | 1280 | 1280 | 1280 | 320 |
| 18 | 30 | F | Hispanic | Pfizer | 126 | 43 | 2560 | 2560 | 1280 | 1280 | 1280 | 453 |
| 19 | 33 | F | Asian | Moderna | 326 | 48 | 2560 | 2560 | 1280 | 1810 | 1280 | 320 |
| 20 | 28 | M | Hispanic | Pfizer | 159 | 60 | 3620 | 640 | 640 | 640 | 320 | 160 |
| 21 | 74 | M | White | Pfizer | 313 | 118 | 3620 | 1280 | 640 | 640 | 640 | 160 |
| 22 | 66 | M | White | Pfizer | 156 | 16 | 5120 | 3620 | 1280 | 2560 | 1280 | 640 |
| 23 | 59 | M | White | Pfizer | 239 | 18 | 5120 | 7241 | 2560 | 5120 | 5120 | 2560 |
| 24 | 18 | F | Hispanic | Pfizer | 309 | 18 | 5120 | 5120 | 1280 | 2560 | 1810 | 640 |
| 25 | 35 | M | White | Moderna | 303 | 63 | 5120 | 5120 | 2560 | 1280 | 2560 | 320 |
| 26 | 63 | F | Hispanic | Pfizer | 279 | 36 | 7241 | 5120 | 2560 | 2560 | 2560 | 1280 |
| 27 | 51 | M | Asian | Pfizer | 391 | 28 | 10240 | 10240 | 5120 | 10240 | 5120 | 1810 |
| 28 | 71 | F | White | Pfizer | 346 | 39 | 14482 | 7241 | 3620 | 5120 | 5120 | 640 |
| 29 | 46 | F | Hispanic | Pfizer | 266 | 29 | 20480 | 20480 | 10240 | 14482 | 10240 | 1280 |
| #GMT | - | - | - | - | - | - | 2114 | 1705 | 730 | 961 | 813 | 274 |
| †95% Cl | - | - | - | - | - | - | 1411-3167 | 1112-2614 | 465-1145 | 612-1509 | 511-1294 | 182-412 |

∗Individual FFRNT₅₀ value is the geometric mean of duplicate plaque assay results.
#Geometric mean neutralizing titers (GMT).
†95% confidence interval (95% CI) for the GMT.

TABLE 5

Thirty-eight human serum samples collected after 3 doses of mRNA vaccine and a subsequent Omicron BA.1 breakthrough infection, Related to FIG. 8

| Sample ID | Age (year) | Gender (M/F) | Ethnicity | Vaccine type | COVID test positive days post last vaccine | Serum collection time (Post COVID test positive days) | FFRNT₅₀ | | | | | | |
|---|---|---|---|---|---|---|---|---|---|---|---|---|---|
| | | | | | | | USA-WA1/2020 | BA.1-spike | BA.2-spike | BA.2.12.1-spike | BA.3-spike | BA.4/5-spike | BA.2.75-spike |
| 1 | 36 | M | Hispanic | Pfizer | 142 | 91 | 453 | 226 | 160 | 160 | 160 | 40 | 160 |
| 2 | 36 | M | Hispanic | Pfizer | 142 | 61 | 640 | 453 | 160 | 320 | 320 | 40 | 160 |
| 3 | 64 | M | White | Pfizer | 40 | 75 | 640 | 160 | 160 | 160 | 113 | 80 | 80 |
| 4 | 62 | F | White | Pfizer | 130 | 81 | 640 | 453 | 320 | 320 | 320 | 160 | 160 |
| 5 | 27 | M | White | Moderna | 111 | 134 | 640 | 320 | 160 | 320 | 226 | 80 | 160 |
| 6 | 34 | F | Black | Pfizer | 85 | 40 | 1280 | 1280 | 640 | 640 | 640 | 320 | 640 |
| 7 | 60 | F | Hispanic | Pfizer | 39 | 76 | 1280 | 640 | 320 | 320 | 320 | 80 | 453 |
| 8 | 29 | F | White | Pfizer | 15 | 91 | 1280 | 453 | 320 | 320 | 320 | 80 | 320 |
| 9 | 58 | M | Asian | Pfizer | 30 | 113 | 1280 | 1280 | 640 | 1280 | 640 | 453 | 640 |
| 10 | 39 | F | Asian | Pfizer | 110 | 23 | 2560 | 2560 | 1280 | 1280 | 1280 | 320 | 1280 |
| 11 | 72 | M | White | Pfizer | 28 | 32 | 2560 | 2560 | 1280 | 1810 | 1280 | 226 | 2560 |
| 12 | 43 | F | Asian | Pfizer | 89 | 58 | 2560 | 2560 | 2560 | 1810 | 1280 | 905 | 2560 |
| 13 | 45 | F | Hispanic | Pfizer | 141 | 46 | 2560 | 5120 | 1810 | 2560 | 2560 | 640 | 1810 |
| 14 | 51 | F | Black | Pfizer | 44 | 35 | 2560 | 2560 | 905 | 640 | 1280 | 320 | 1280 |
| 15 | 56 | F | Black | Pfizer | 98 | 93 | 2560 | 2560 | 640 | 1280 | 1280 | 226 | 1280 |
| 16 | 67 | F | White | Pfizer | 71 | 63 | 2560 | 1280 | 640 | 640 | 640 | 160 | 640 |
| 17 | 64 | M | White | Pfizer | 114 | 33 | 2560 | 2560 | 1280 | 1280 | 1280 | 320 | 905 |
| 18 | 28 | F | White | Pfizer | 158 | 61 | 2560 | 2560 | 1280 | 1280 | 1280 | 320 | 1810 |
| 19 | 64 | M | Asian | Pfizer | 117 | 73 | 2560 | 2560 | 1280 | 1280 | 1280 | 160 | 2560 |
| 20 | 63 | F | White | Pfizer | 174 | 22 | 2560 | 2560 | 1280 | 2560 | 905 | 1280 | 1280 |
| 21 | 41 | F | Hispanic | Moderna (dose 1-2) & Pfizer (dose 3) | 84 | 64 | 2560 | 1280 | 320 | 453 | 453 | 160 | 640 |
| 22 | 64 | F | Asian | Pfizer | 118 | 105 | 3620 | 2560 | 1280 | 1280 | 1280 | 320 | 2560 |
| 23 | 54 | M | White | Pfizer | 93 | 34 | 5120 | 2560 | 2560 | 2560 | 1810 | 640 | 2560 |
| 24 | 26 | F | White | Moderna | 63 | 51 | 5120 | 5120 | 1810 | 2560 | 2560 | 1280 | 2560 |
| 25 | 44 | F | Hispanic | Pfizer | 43 | 15 | 5120 | 2560 | 1280 | 1280 | 1280 | 640 | 1810 |
| 26 | 77 | M | Hispanic | Pfizer | 130 | 64 | 5120 | 5120 | 2560 | 2560 | 2560 | 1280 | 2560 |
| 27 | 69 | M | White | Pfizer | 124 | 79 | 5120 | 2560 | 1280 | 1810 | 1280 | 320 | 1280 |
| 28 | 73 | F | White | Moderna | 164 | 36 | 5120 | 3620 | 2560 | 1810 | 1280 | 226 | 640 |
| 29 | 84 | F | Hispanic | Pfizer | 99 | 46 | 5120 | 5120 | 1280 | 2560 | 1280 | 226 | 2560 |
| 30 | 61 | F | White | Pfizer | 169 | 40 | 5120 | 2560 | 640 | 1280 | 905 | 226 | 1280 |
| 31 | 58 | M | Hispanic | Moderna | 77 | 73 | 5120 | 1810 | 640 | 905 | 1280 | 320 | 1280 |
| 32 | 75 | M | White | Pfizer | 131 | 108 | 5120 | 2560 | 1280 | 1280 | 1280 | 320 | 1280 |
| 33 | 79 | F | White | Pfizer | 144 | 17 | 5120 | 2560 | 640 | 1280 | 905 | 640 | 1280 |
| 34 | 38 | M | White | Pfizer | 109 | 49 | 10240 | 5120 | 5120 | 5120 | 2560 | 1280 | 2560 |
| 35 | 71 | M | White | Pfizer | 129 | 54 | 10240 | 7241 | 2560 | 2560 | 2560 | 80 | 5120 |
| 36 | 39 | F | Hispanic | Pfizer | 118 | 126 | 10240 | 5120 | 2560 | 3620 | 2560 | 640 | 2560 |
| 37 | 68 | M | White | Pfizer | 125 | 71 | 10240 | 5120 | 2560 | 2560 | 2560 | 640 | 80 |
| 38 | 66 | M | White | Pfizer | 96 | 41 | 20480 | 20480 | 10240 | 14482 | 10240 | 1810 | 10240 |
| #GMT | - | - | - | - | - | - | 2962 | 2038 | 983 | 1190 | 1019 | 297 | 1019 |
| †95% Cl | - | - | - | - | - | - | 2212-3967 | 1462-2841 | 712-1357 | 869-1630 | 759-1367 | 216-410 | 702-1480 |

∗Individual FFRNT₅₀ value is the geometric mean of duplicate plaque assay results.
#Geometric mean neutralizing titers (GMT).
†95% confidence interval (95% Cl) for the GMT.

### B. Methods

Ethical statement. The work was performed in a biosafety level 3 (BSL-3) laboratory with redundant fans in the biosafety cabinets at The University of Texas Medical Branch at Galveston. All personnel wore powered air-purifying respirators (Breathe Easy, 3M) with Tyvek suits, aprons, booties, and double gloves.

The research protocol regarding the use of human serum specimens was reviewed and approved by the University of Texas Medical Branch (UTMB) Institutional Review Board (IRB number 20-0070). No informed consent was required since these deidentified sera were leftover specimens before being discarded. No diagnosis or treatment was involved.

Cells. Vero E6 (ATCC® CRL-1586) was purchased from the American Type Culture Collection (ATCC, Bethesda, MD), and maintained in a high-glucose Dulbecco’s modified Eagle’s medium (DMEM) containing 10% fetal bovine serum (FBS; HyClone Laboratories, South Logan, UT) and 1% penicillin/streptomycin at 37°C with 5% CO₂. Culture media and antibiotics were purchased from ThermoFisher Scientific (Waltham, MA). The cell line was tested negative for mycoplasma.

Human Serum. Three panels of human sera were used in the study. The first panel consisted of 25 pairs of sera collected from individuals 3-8 months after vaccine dose 3, and no more than 3 months after dose 4 of Pfizer or Moderna vaccine. This panel had been tested negative for SARS-CoV-2 nucleocapsid protein expression using Bio-Plex Pro Human IgG SARS-CoV-2 N/RBD/S1/S2 4-Plex Panel (Bio-rad). The second serum panel (n=29) was collected from individuals who had received 2 doses of mRNA vaccine and subsequently contracted Omicron BA.1. The third serum panel (n=38) was collected from individuals who had received 3 doses of mRNA vaccine and subsequently contracted Omicron BA.1. The genotype of infecting virus was verified by the molecular tests with FDA’s Emergency Use Authorization and Sanger sequencing. The de-identified human sera were heat-inactivated at 56°C for 30 min before the neutralization test. The serum information is presented in Table S1-3.

Recombinant Omicron sublineage spike mNG SARS-CoV-2. Recombinant Omicron sublineage BA.1-, BA.2-, BA.2.12.1-, BA.3-, BA.4/5-spike mNG SARS-CoV-2s that was constructed by engineering the complete spike gene from the indicated variants into an infectious cDNA clone of mNG USA-WA1/2020 were reported previously (Kurhade et al., 2022b; Xie et al., 2020). BA.2.75-spike sequence was based on GISAID EPI_ISL_13521499. FIG. 7A depicts the spike mutations from different Omicron sublineages. The full-length cDNA of viral genome bearing the variant spike was assembled via in vitro ligation and used as a template for in vitro transcription. The full-length viral RNA was then electroporated into Vero E6-TMPRSS2 cells. On day 3-4 post electroporation, the original P0 virus was harvested from the electroporated cells and propagated for another round on Vero E6 cells to produce the P1 virus. The infectious titer of the P1 virus was quantified by fluorescent focus assay on Vero E6 cells and sequenced for the complete spike gene to ensure no undesired mutations. The P1 virus was used for the neutralization test. The protocols for the mutagenesis of mNG SARS-CoV-2 and virus production were reported previously (Hachmann et al., N Engl J Med 387, 86-88, 2022).

Fluorescent focus reduction neutralization test. A fluorescent focus reduction neutralization test (FFRNT) was performed to measure the neutralization titers of sera against USA-WA1/2020, BA.1-, BA.2-, BA.2.12.1-, BA.3-, and BA4/5-spike mNG SARS-CoV-2. The FFRNT protocol was reported previously (Zou et al., 2022a). Vero E6 cells were seeded onto 96-well plates with 2.5×10⁴ cells per well (Greiner Bio-one™) and incubated overnight. On the next day, each serum was 2-fold serially diluted in a culture medium and mixed with 100-150 focus-forming units of mNG SARS-CoV-2. The final serum dilution ranged from 1:20 to 1:20,480. After incubation at 37°C for 1 h, the serum-virus mixtures were loaded onto the pre-seeded Vero E6 cell monolayer in 96-well plates. After 1 h infection, the inoculum was removed and 100 µl of overlay medium containing 0.8% methylcellulose was added to each well. After incubating the plates at 37°C for 16 h, raw images of mNG foci were acquired using Cytation™ 7 (BioTek) armed with 2.5× FL Zeiss objective with a wide field of view and processed using the software settings (GFP [469,525] threshold 4000, object selection size 50-1000 µm). The fluorescent mNG foci were counted in each well and normalized to the non-serum-treated controls to calculate the relative infectivities. The FFRNT₅₀ value was defined as the minimal serum dilution to suppress >50% of fluorescent foci. The neutralization titer of each serum was determined in duplicate assays, and the geometric mean was taken. Tables 3-5 summarize the FFRNT₅₀ results.

1. A recombinant DNA expression cassette comprising a recombinant SARS-CoV-2 nucleic acid segment encoding a heterologous S protein and a reporter protein replacing an ORF7a encoding segment.

2. The expression cassette of claim 1, wherein the heterologous S protein is a variant of SEQ ID NO:2.

3. The expression cassette of claim 1, wherein the nucleic acid segment encoding the heterologous S protein has a nucleic acid sequence that is at least 98% identical to the nucleic acid sequence of SEQ ID NO:2.

4. The expression cassette of claim 1, wherein the nucleic acid encoding the heterologous S protein has a nucleic acid sequence of SEQ ID NO:2.

5. The expression cassette of claim 1, wherein the encoded heterologous S protein has an amino acid sequence that is at least 98% identical to SEQ ID NO:3.

6. The expression cassette of claim 1, wherein the encoded heterologous S protein has an amino acid sequence of SEQ ID NO:3.

7. The expression cassette of claim 1, wherein the recombinant SARS-CoV-2 nucleic acid segment is at least 95% identical to the nucleic acid sequence of SEQ ID NO:1.

8. The expression cassette of claim 1, wherein the SARS-CoV-2 nucleic acid segment is at least 99% identical to the nucleic acid sequence of SEQ ID NO:1.

9. The expression cassette of claim 1, wherein the SARS-CoV-2 nucleic acid segment has a nucleic acid sequence of SEQ ID NO:1.

10. The expression cassette of claim 1, wherein the expression cassette is comprised in a plasmid backbone.

11. The expression cassette of claim 1, wherein the SARS-CoV-2 nucleic acid segment is operatively coupled to a heterologous promoter segment.

12. (canceled)

13. A recombinant SARS-CoV-2 genome comprising a nucleic acid sequence encoding a heterologous S protein and a reporter protein replacing an ORF7a encoding segment.

14. The recombinant SARS-CoV-2 of claim 13, wherein the reporter protein is a fluorescent or luminescent protein.

15. The recombinant SARS-CoV-2 of claim 14, wherein the fluorescent protein is mNeonGreen protein.

16. The recombinant SARS-CoV-2 of claim 14, wherein the luminescent protein is nanoluciferase protein.

17. A recombinant cDNA comprising a nucleic acid sequence encoding a heterologous S protein and a reporter protein replacing an ORF7a.

18. The recombinant cDNA of claim 17, wherein the heterologous S protein is a variant of SEQ ID NO:2.

19. The recombinant cDNA wherein the nucleotide sequence is 95, 96, 97, 98, 99 to 100% identical to SEQ ID NO:1.

20. An assay for SARS-CoV-2 replication comprising:
contacting a cultured cell expressing or containing a SARS-CoV-2 nucleotide sequence of claim 1 forming a test cell;
contacting the test cell with a test agent; and
assessing the replication of the SARS-CoV-2 in the presence of the test agent.

21. The assay of claim 20, wherein the cultured cell is a Vero cell.

22-24. (canceled)
